(19) United States
(12) Patent Application Publication
(10) Pub. No.: US 2003/0219402 A1
(43) Pub. Date: Nov. 27, 2003

(54) Chimeric molecules for cleavage in a treated host

(76) Inventor: William J. Rutter, San Francisco, CA (US)

Correspondence Address: COOLEY GODWARD LLP, ATTN: PATENT GROUP, 11951 FREEDOM DRIVE, SUITE 1700, ONE FREEDOM SQUARE- RESTON TOWN CENTER, RESTON, VA, 20190-5061, US

(21) Appl. No.: 10/367,555

(22) Filed: Feb. 14, 2003

Related U.S. Application Data
(60) Provisional application No. 60/357,740, filed on Feb. 14, 2002.

(51) Int. Cl. A61K 38/19; A61K 48/00; A61K 38/14; A61K 31/715; A61K 39/00; A61K 35/84; A61K 38/43; A61K 35/78

(52) U.S. Cl. 424/85.1; 514/2; 514/8; 514/44; 514/54; 424/185.1; 424/725; 424/195.15; 424/94.1

(57) ABSTRACT

The present invention relates to chimeric molecules containing component molecules that are linked together in a non-naturally occurring manner where the linker contains at least one enzyme cleavage site, and the enzyme cleavage site is engineered to be cleaved by an enzyme in a treated subject. The present invention also relates to compositions and kits containing the chimeric molecules, methods of making the chimeric molecules in a production host, methods of using the present chimeric molecules for diagnostic, prophylactic, therapeutic, and nutritional purposes in subjects requiring such.

This application is related to provisional application No. 60/357,740 entitled “Chimeric molecules for Cleavage in a Treated Host,” filed Feb. 14, 2002, from which priority is claimed under 35 USC §119(e)(1) and which application is incorporated herein by reference in its entirety.

### FIELD OF THE INVENTION

The present invention relates to the field of chimeric molecules that are suitable for administration to a host for in vivo cleavage to produce a diagnostic, prophylactic, therapeutic and/or nutritional effect in the treated host.

### BACKGROUND OF THE INVENTION

Chimeric molecules have been made for the purpose of in vitro cleavage in the production and purification of recombinant proteins in microbial hosts. U.S. Pat. No. 4,769,326 (the “'326 patent”), entitled “Expression Linkers,” assigned to the Regents of the University of California, issued on Sep. 6, 1988 relates to, among other things, a recombinant DNA sequence “which comprises three segments not contiguous in the natural environment, wherein a first segment encodes a eucaryotic protein and is contiguous with a second segment that encodes a specific cleavage sequence of at least two amino acids, said second segment being contiguous with a third segment, wherein: the expression product of said DNA is specifically cleaved by at least one enzymatic or chemical reagent at the peptide bond linking the eucaryotic protein and the specific cleavage sequence; and the third segment encodes a host peptide wherein the third segment encodes a host peptide wherein the peptide is . . . not natively associated with said eucaryotic protein.” (claim 1)

Since then, others have developed other systems for in vitro production and processing. Examples of such adaptations include the following.

U.S. Pat. No. 4,745,069, issued May 17, 1988, assigned to Eli Lilly & Company, entitled “Cloning Vectors for Expression of Exogenous Protein,” relates to, among other things, “a recombinant DNA cloning vector useful for expressing exogenous protein,” where the cloning vectors were “constructed to contain, in tandem, a nucleotide sequence defining the lipoprotein promoter region, a nucleotide sequence defining the lipoprotein 5′ untranslated region, and a sequence coding for an exogenous protein product, the sequence coding for such product being connected via a translation start signal codon and a nucleotide sequence coding for an enterokinase cleavage site to the 3′ terminal of the 5′ untranslated region of the lipoprotein gene.” (col. 2, lines 45-62) The rationale for this invention appears to be that the “high level of constitutive transcription observed for the lipoprotein gene . . . recommends it as a vehicle for expression of exogenous DNA fragments.” (Col. 2, lines 14-17)

U.S. Pat. No. 4,828,988, issued May 9, 1989, assigned to Smith Kline-RIT, entitled “Hybrid Polypeptides Comprising Somatocrinine and Alpha₁-Antitrypsin, Method for Their Production from Bacterial Clones and Use Thereof for the Production of Somatocrinine,” relates to, among other things, “expression of hGRF in bacteria [which] can be significantly and unexpectedly improved by fusing the coding sequence for hGRF to a coding sequence corresponding to hAT or a fragment thereof which expresses itself in an optimal manner in bacteria . . . .” (Col. 3, lines 4-9)

U.S. Pat. No. 5,292,646 (the “'646 patent”), issued Mar. 8, 1994, assigned to Genetics Institute, Inc., entitled “Peptide and Protein Fusions to Thioredoxin and Thioredoxin-like Molecules,” relates to, among other things, “a fusion sequence comprising a thioredoxin-like protein sequence fused to a selected heterologous peptide or protein,” where the fusion sequence “may optionally contain a linker peptide,” which provides “where needed, a selected cleavage site . . . ” (col. 2, lines 47-60). The invention described in the '646 patent aims to provide “a novel method for increasing the expression of soluble recombinant proteins,” which method includes “culturing under suitable conditions the above-described host cell to produce the fusion protein.” (Col. 3, lines 6-10)

U.S. Pat. No. 6,080,559, issued Jun. 27, 2000 to Agennix, Inc., entitled “Expression of Processed Recombinant Lactoferrin and Lactoferrin Polypeptide Fragments from a Fusion Product in Aspergillus,” relates to “An intact, deglycosylated lactoferrin protein or a single domain, deglycosylated lactoferrin polypeptide fragment produced by a process that comprises culturing a transformed Aspergillus fungal cell containing a recombinant plasmid, wherein said plasmid comprises the following components operably linked from 5′ to 3′: (a) a promoter; (b) a nucleotide sequence encoding a signal peptide; (c) a 5′ portion of a nucleotide sequence of a gene encoding an amino-terminal portion of a highly expressed endogenous, secreted Aspergillus polypeptide; (d) a nucleotide sequence encoding a peptide linker, said peptide linker comprising a cleavage site of a protease endogenous to Aspergillus; and (e) a nucleotide sequence encoding lactoferrin or lactoferrin polypeptide fragment; wherein said transformed Aspergillus fungal cell is cultured in a suitable nutrient medium until a lactoferrin protein or a lactoferrin polypeptide fragment is produced as a fusion product and then processed via an endogenous proteolytic enzyme specific for said linker sequence, wherein said processed lactoferrin or lactoferrin polypeptide fragment is secreted into the nutrient medium and isolated therefrom and wherein the lactoferrin protein or the lactoferrin polypeptide fragment has been deglycosylated.” (claim 1)

WO 97/28272, filed by Technologene, Inc., published Aug. 7, 1997, entitled “Protein Expression System,” relates to, among other things, “methods for expression and purification of authentic recombinant proteins from such fusion proteins. In particular, the present invention relates to fusion proteins wherein additional domains and/or elements are added to the fusion proteins. Included in these domains and/or elements are Fc fragments (1) fused to proteins of interest (s) by a polypeptide comprising a hinge region (3), hydrophilic spacer (4), and a dibasic amino acid endoprotease cleavage site (5), wherein the spacer may be cleaved and then digested by carboxypeptidase B (6) to yield the authentic protein (2).” (Abstract)

WO 99/58662, a Japanese application published May 13, 1995, entitled “Fused Protein” relates to a fused protein containing “a target protein which consists of, in the direction from the N-end towards the C-end, a) a signal sequence, b) an immunoglobulin Fc region with the deletion of at least CH1 domain; c) a peptide linker containing at the C-end an enzyme cleavage site allowing cleaving with enterokinase, etc.; and d) the amino acid sequence of the target protein such as erythropoietin, characterized in that, after the completion of enzymatic cleavage, the target protein contains at the N-end no amino acid residue originating in the peptide linker. Thus, a target protein free from any amino acid-modification at the N-end can be efficiently produced by an enzymatic treatment.” (Abstract)

WO00/23472, filed by Biogen, Inc., entitled “Interferon-Beta Fusion Proteins and Uses,” relates to, among other things, “an interferon-beta-la composition with increased activity relative to interferon-beta-1b and that also has the salutary properties of fusion proteins in general with no effective loss in activity . . . .” (Page 2, lines 17-19). The specification describes this invention as relating to “an isolated polypeptide having the amino acid sequence X-Y-Z, wherein X is a polypeptide having an amino acid sequence, or portion thereof, consisting of the amino acid sequence of interferon beta; Y is an optional linker moiety; and Z is a polypeptide comprising at least a portion of a polypeptide other than interferon beta.” (Page 3, lines 1-5) This invention further relates to “a method of producing a recombinant polypeptide comprising: providing a population of host cells according to the invention; growing the population of cells under conditions whereby the polypeptide encoded by the recombinant DNA is expressed; and isolating the expressed polypeptide.” (Page 4, lines 3-6)

WO00/39310, filed by The University of Georgia Research Foundation, entitled “Rubredoxin Fusion Proteins, Protein Expression System and Methods,” relates to, among other things, “a recombinant rubredoxin fusion protein containing an N-terminal rubredoxin constituent and a C-terminal fusion polypeptide.” (at page 3, lines 3-5) This fusion protein “is capable of binding Fe²⁺ when properly folded, giving it a red: color that makes it easy to follow during purification.” (page 3, lines 5-6) “The linkage between the N-terminal rubredoxin constituent and C-terminal fused polypeptide can, but need not, be a cleavable linkage.” (page 3, lines 15-17)

WO00/61768, filed by Yeda Research and Development Co., Ltd., entitled “Preparation of Biologically Active Molecule,” relates to, among other things, “the production of molecules which, in their natural process of formation are produced in a biologically inactive form, and become active after cleavage of their precursor.” The specification provides that in a preferred embodiment, “the method comprises transfecting a host with a vector comprising a cDNA encoding a precursor of a biologically active molecule mutated at its cleavage site, culturing the transfected host, expressing the precursor and isolating the biologically active molecule after treatment with a protease.” (Page 3, lines 27-31)

WO01/14570, filed by Allergan Sales, Inc., entitled “Activable Recombinant Neurotoxins” relates to, among other things, “recombinant and isolated proteins comprising a functional binding domain, translocation domain, and therapeutic domain in which such proteins also include an amino acid sequence that is susceptible to specific cleavage in vitro following expression as a single chain” (at page 7, lines 19-22). The translocation element therein “comprises a portion of a clostridial neurotoxin H chain having a translocation activity” (at page 19, lines 11-12). This invention addresses the issue of the “degree of activation of engineered clostridial toxins” as “an important consideration for manufacture of these materials.” Hence, it would be “a major advantage if neurotoxins such as BoNT [botulinum neurotoxin] and TeTx [tentanus neurotoxin] could be expressed in high yield in rapidly-growing bacteria (such as heterologous E. coli cells) as relatively non-toxic single chains (or single chains having reduced toxic activity) which are safe, easy to isolate and simple to convert to fully-active form.” (Page 6, lines 5-11)

In vivo production of a fused molecule coupled with in vitro processing of the molecule is described in Proc. Natl. Acad. Sci USA 91(20): 9337-41 (Sep. 27, 1994), entitled “High-efficiency synthesis of human alpha-endorphin and magainin in the erythrocytes of transgenic mice: a production system for therapeutic peptides.”

In certain instances, it may be desirable to express polypeptides in vivo, rather than delivering polypeptides synthesized in vitro. As an example, polypeptides synthesized by a host in vivo may undergo advantageous post-translational modifications, such as, amidation of the C-terminus or glycosylation. (See U.S. Pat. No. 5,707,826 issued Jan. 13, 1998 to BioNebraska, Inc., col. 1, lines 18-25; and EP 0134 085, published Mar. 13, 1985, filed by the Salk Institute for Biological Studies, at page 3, lines 13-16; page 4, lines 3-5).

Thus, methods of gene therapy have been applied to deliver therapeutic genes into organisms for production of protein in vivo. Yet the reality of gene therapy is still far away, and gene therapy as a form of treatment has yet to be approved. Examples of gene therapy delivery of nucleic acids include the following:

U.S. Pat. No. 6,228,356, issued May 8, 2001 to the University of Pittsburgh of the Commonwealth System of Higher Education, entitled “Viral Vectors to Inhibit Leukocyte Infiltration or Cartilage Degradation of Joints,” relates to, among other things, a method “for inhibiting leukocyte infiltration or cartilage degradation in a joint of a mammal, the method comprising directly administering to a said joint a viral vector comprising a nucleic acid sequence, operably linked to a promoter, encoding a protein that counteracts an effect of IL-1 in a joint, wherein expression of said protein within said joint results in an inhibition of leukocyte infiltration or cartilage degradation in said joint.” (claim 1) The proteins that counteract the effect of IL-1 are, for example, an interleukin-1 receptor antagonist protein (IRAP) (claim 2), a soluble interleukin-1 receptor (claim 3), a soluble TNF receptor (claim 4), or interleukin-10 (claim 5). Further disclosed is a method of producing an animal model for study of connective tissue pathology where the method “includes employing as the gene a material selected from the group consisting of a cytokine and a proteinase,” where the proteinase employs a matrix metalloproteinase, and the matrix metalloproteinase is “selected from the group consisting of a collagenase, a gelatinase, and a stromelysin.” (Col. 14, line 36 to col. 15, line 1) Although the specification provides for “introducing at least one gene encoding a product into at least one cell of a connective tissue of a mammalian host” (Abstract), no particular strategy is apparent for the delivery of more than one gene at a time. Related to this is U.S. Pat. No. 5,858,355 entitled “IRAP gene as treatment for arthritis,” issued Jan. 12, 1999. which discloses transfecting synovial cells in vitro and transplanting the infected synovial cells by intraarticular injection to an arthritic joint space, to cause reduction of cartilage destruction or reduction in synovitis.

U.S. Pat. No. 6,017,896, issued on Jan. 25, 2000, to University of Alabama Research Foundation and Southern Research Institute, entitled “Purine Nucleoside Phosphorylase Gene Therapy for Human Malignancy,” relates to, among other things, a method “of killing replicating or non-replicating, transfected or transduced mammalian cells and bystander cells,” by “(a) transfecting or transducing mammalian cells with a nucleic acid encoding a purine cleavage enzyme capable of cleaving an adenosine; and (b) contacting the transfected or transduced cells with an effective amount of a substrate for the purine cleavage enzyme, wherein the substrate is substantially non-toxic to mammalian cells and is cleaved by the enzyme to yield a purine toxic to transfected or transduced mammalian cells and bystander cells, to kill the mammalian cells expressing the enzyme and the bystander cells.” (claim 1)

U.S. Pat. No. 6,080,575, issued Jun. 27, 2000, to Hoechst Aktiengeselschaft AG, entitled “Nucleic Acid Construct for Expressing Active Substances which can be Activated by Proteases, and Preparation and Use,” relates to, among other things, “a nucleic acid construct” which is “activated by an enzyme which is released from mammalian cells, which construct comprises the following components: a) at least one promoter element, b) at least one DNA sequence which encodes an active compound (protein B) c) at least one DNA sequence which encodes an amino acid sequence (part structure C) which can be cleaved specifically by an enzyme which is released from a mammalian cell, and d) at least one DNA sequence which encodes a peptide or protein (part structure D) which is bound to the active compound (protein B) by way of the cleavable amino acid sequence (part structure C) and inhibits the activity of the active compound . . . .” (Abstract)

U.S. Pat. No. 6,147,055, issued Nov. 14, 2000 to Vical Incorporated, entitled “Cancer Treatment Method Utilizing Plasmids Suitable for IL-2 Expression,” relates to, among other things, “a method for treating cancer in a human patient, comprising: administering in vivo directly into a tumor of said patient a DNA plasmid formulated with a cationic lipid; wherein said plasmid comprises (1) a first polynucleotide encoding a mature human interleukin 2 (IL-2) polypeptide; (2) a second polynucleotide encoding a peptide leader operably linked to said first polynucleotide, wherein said peptide leader directs secretion of said IL-2; and (3) a promoter operably associated with said first and second polynucleotides . . . .” (claim 1)

Foreign genes have also been introduced into and expressed in plants. For example, U.S. Pat. No. 5,939,541, issued on Aug. 17, 1999 to University of South Carolina, entitled “Method for Enhancing Expression of a Foreign Gene or Endogenous Gene Product in Plants,” relates to, among other things, the provision of “a booster sequence comprising the coding region for P1, helper component-proteinase (HC-Pro) and a portion of P3, so that said booster sequence includes the region encoding the protein cleavage site required for autoproteolytic processing of the HC-Pro carboxy-terminus of the genome of a potyvirus to said plant cells, plant protoplasts, or whole plants so that expression of said foreign gene or endogenous plant gene is enhanced as compared to said expression in said plant cells, plant protoplasts, or whole plants without said booster sequence.” (claim 1)

U.S. Pat. No. 5,491,076, issued on Feb. 13, 1996 to Texas A&M University System, entitled “Expression of Foreign Genes Using a Replicating Polyprotein Producing Virus Vector,” relates to, among other things, “an expression vector adapted for expressing heterologous proteins in plants susceptible to a polyprotein-producing plant virus. The vector utilizes the unique ability of viral polyprotein proteases to cleave heterologous proteins from viral polyproteins.” (Abstract) Notably, the vector comprises cDNA, “wherein said cDNA comprises sequences that code for a replicatable genome of a polyprotein-producing Tobacco Etch Virus” (claims 1 and 2).

WO00/11175A1 published Mar. 2, 2000, filed by Zeneca Limited, and entitled “Genetic Method for the Expression of Polyproteins in Plants,” relates to, among other things, “a method of expressing or improving expression levels of one or more proteins in a transgenic plant comprising inserting into the genome of said plant a DNA sequence comprising a promoter region operably linked to two or more protein encoding regions and a 3′-terminator region wherein said protein encoding regions are separated from each other by a DNA sequence coding for a linker propeptide, said propeptide providing a cleavage site whereby the expressed polyprotein is post-translationally processed into the component protein molecules. In particular, a signal sequence is also included such that the post-translational processing is effected in the secretory pathway of plants. Suitable linker sequences and DNA constructs for use in the method are also described.” (Abstract)

U.S. Pat. No. 5,912,167, issued Jun. 15, 1999, filed by Wisconsin Alumni Research Foundation, entitled “Autocatalytic Cleavage Site and Use Thereof in a Protein Expression Vector,” relates to, among other things, “a method of using the autocatalytic cleavage site found in picomaviruses to usefully express a recombinant peptide or protein. (Col. 3, lines 24-26) Also disclosed is “a nucleic acid construct comprising at least two copies of a nucleic acid sequence encoding an autocatalytic peptide cleavage site,” where the site comprises a specified amino acid sequence and “wherein the construct is part of a replication competent picomavirus viral sequence and wherein the copies are located at the site of a naturally occurring autocatalytic cleavage site.” (claim 1). Claim 4 depends from claim 1 and recites a polylinker that is “between two copies of the nucleic acid sequence encoding the autocatalytic site.” claim 6 depends from claim 5, which is dependent from claim 4, and describes the amino acid sequence encoded by the nucleic acid sequence to comprise “a polyprotein, wherein the polyprotein comprises heterologous proteins that are separated by the autocatalytic cleavage sites.” In another dependent claim, the vector of claim 1 is a Mengo virus. (claim 7)

U.S. Pat. No. 6,221,355, issued Apr. 24, 2001, to Washington University, entitled “Anti-pathogen System and Methods of Use Thereof,” relates to the use of “one or more fusion proteins that includes a transduction domain and a cytotoxic domain. The cytotoxic domain is specifically activated by a pathogen infection. The anti-pathogen system effectively kills or injures cells infected by one or a combination of different pathogens.” (Abstract)

WO 98/13059, filed by Bristol-Myers Squibb Co., entitled “Hydrolyzable Prodrugs for Delivery of Anticancer Drugs to Metastatic Cells,” relates to hydrolysable prodrugs that “are activated by proteases located in the cell membranes of metastatic cells to yield active anticancer drugs that can be taken up by the metastatic cells. In general, a hydrolysable prodrug according to the present invention comprises an amino-terminal capped peptide that is a substrate for a peptidohydrolase located on the surface of a metastatic cell covalently linked to a therapeutic drug through a self-immolating spacer of sufficient length to prevent the occurrence of steric hindrance. The therapeutic drug is typically an anticancer drug. The anticancer drug is typically doxorubicin, taxol, camptothecin, mitomycin C, or esperamycin. Typically, the peptidohydrolase that hydrolyses the substrate of the hydrolysable prodrug is cathepsin B.” (Abstract)

U.S. Pat. No. 6,251,392 issued to Epicyte Pharmaceuticals, Inc. on Jun. 26, 2001, entitled “Epithelial Cell Targeting Agent,” relates to targeting molecules “for use in delivering biological agents to non-polarized epithelial cells,” where upon delivery, “the biological agent(s) are lethal to epithelial cell. The targeting molecules may be used, for example, for the eradication of metastatic epithelial cells.” (Abstract)

Many diseases or conditions are associated with the presence or lack thereof of more than one gene product. Thus, administration of a medication with one active component to address the presence or lack of more than one gene product may not be sufficient to control the disease or condition very effectively. Furthermore, if the half life of a medication can be lengthened, it may be possible to administer the medication fewer than several times a day, a preferred regimen for many reasons, including convenience, cost, and safety. Additionally, it would be desirable if the dose of a given medication could be reduced and, thus, its side effects diminished if the medication can be more effectively delivered to a locale where action is needed. Furthermore, it would be desirable if there is a method of delivery of active molecules that is cost-effective, without the high cost associated with purification of a molecule to over 90% purity for delivery to a treated host. Thus, there is an unmet need for a more effective and efficient delivery of one or more active molecules to a desired site in a treated host.

There is further a recognition for need of an expression system for production of small molecules, such as recombinant small molecule peptides, that would not be degraded in the host producing such peptides.

### SUMMARY OF THE INVENTION

It is one of the objects of the present invention to address the unmet needs in the art, as stated above.

It is a further one of the objects of the present invention to provide a method for delivery of a plurality of component molecules at one time to a multi-cellular host (“treated host”), for diagnostic, therapeutic, prophylactic or nutritional purposes.

It is also another one of the objects of the present invention to provide a method for delivery of molecules to a treated host that would normally be degraded in the treated host.

It is yet another one of the objects of the present invention to deliver molecules to a site of action in the treated host to maximize the effect of the molecules and to minimize side effects.

In accordance to one of the objects, there is provided method of delivery of a plurality of component molecules to a multi-cellular host, comprising the steps of: (a) providing a composition comprising a chimeric molecule; and (b) administering the chimeric molecule to the host to produce a treated host, wherein the chimeric molecule comprises at least one first component molecule, at least one linker, and at least one second component molecule; wherein the linker comprises an enzyme cleavage site and wherein at least a first linker is operably linked to a first component molecule and a second component molecule to produce a non-naturally occurring linkage and cleavage site between the first component molecule and second component molecule; wherein the cleavage site is engineered for cleavage in vivo by a host enzyme and is resistant to cleavage in any production host; wherein, upon cleavage of the chimeric molecule at the cleavage site, at least one of the component molecules is functionally active; and wherein at least one of the first and second component molecules comprises one selected from the group consisting of a peptide, a protein, or an active fragment thereof.

In accordance to another one of the objects, there is provided a method as above, where the cleavage site is engineered for cleavage in vivo by an enzyme that is localized on an enzyme that circulates systemically. Thus, for example, the cleaving enzyme may be localized in the alimentary tract, genitourinary tract, tears, saliva, and the like. The cleaving enzyme may also be present systemically. In one embodiment, the cleaving enzyme is present in the gastrointestinal tract of the host, such as, for example, any digestive enzyme, including any of the enteropeptidases, such as trypsin, chymotrypsin, elastase, enterokinase, or by a tissue type plasminogen activator, such as involved in the process of metastasis, and matrix metalloproteinase, which is similarly involved.

In accordance to yet another one of the objects of the present invention, there is provided a method as above, where the cleavage site is engineered for cleavage in vivo extracellularly in the treated host, other than at a cell surface, for example, where one or both of the component molecules is not or other than an interferon-β.

In accordance to yet another one of the objects of the present invention, there is provided a method as above, where the cleavage site is engineered for cleavage in vivo in the treated host at a cell surface, for example, where the first component molecule is not or other than an antibody or an antibody fragment.

In accordance to a further one of the objects, there is provided a method as above, where the cleavage site is engineered for cleavage by an endogenous treated host enzyme. In one embodiment, the cleavage site is engineered for cleavage by an endogenous host enzyme selected from the group consisting of: coagulation factors; ADAMTS 4 and 5; Aggreganases 1 and 2; thrombin; plasmin; complement factors; gastricin; granule proteases; matrix metalloproteinases; membrane type matrix metalloproteinases; type II transmembrane serine proteases; ADAMs; neprilysin; urokinase-type plasminogen activator, tissue type plasminogen activator and caspases.

In accordance to a further one of the objects, there is provided a method as above, where the cleavage site is engineered for cleavage in vivo intracellularly by an enzyme in the treated host, and the combination of first and second component molecules is other than the combination of a protein transduction domain and a cytotoxic domain.

In accordance to still another one of the objects, there is provided a method as above, where the cleavage site is engineered for cleavage in vivo intracellularly by an enzyme in the treated host, and the cleavage site is not a viral pathogen activated cleavage site.

In accordance to yet another one of the objects, there is provided a method as above, where the cleavage site is engineered for cleavage in vivo intracellularly by an enzyme in the treated host, and the second component is not or other than a cytotoxic molecule.

In accordance to another one of the objects, there is provided a method as above, where upon cleavage of the chimeric molecule at the enzyme cleavage site, at least two of the component molecules are functionally active.

In accordance to still another one of the objects, there is provided a method as above, where at least one of the component molecules is functionally active prior to cleavage of the chimeric molecule.

In one embodiment, the component molecules as above are non-inhibitory molecules. In another embodiment, the component molecules are non-cytotoxic molecules. In certain embodiments, the first and second component molecules are the same. In other embodiments, the first and second component molecules are different.

In accordance to another one of the objects, there is provided a method as above where the chimeric molecule has a formula: A(xᵢBᵢ)ⁿ, wherein A represents the first component molecule, x represents the linker, B represents the second component molecule, i and n are each a positive integer.

In accordance to another one of the objects, there is provided a method as above where the formula is selected from the group consisting of:

(a) A(x₁B₁);

(b) A(x₁B₁)(x₂B₂), where x₁ and x₂ may be the same or different, and B₁ and B₂ may be the same or different, and A may be the same or different from B₁ and B₂;

(c) A(x₁B₁)(x₂B₂)(x₃B₃), wherein x₁, x₂ and x₃ may each be the same or different, and B₁, B₂ and B₃ may each be the same or different, and A may be the same or different from B₁, B₂ and B₃;

(d) A(x₁B₁)(x₂B₂)(x₃B₃)(x₄B₄), wherein x₁, x₂, x₃ and x₄ may each be the same or different, and B₁, B₂, B₃ and B₄ may each be the same or different, and A may be the same or different from B₁, B₂, B₃ and B₄; and

(e) A(x₁B₁)(x₂B₂)(x₃B₃)(x₄B₄)(x₅B₅), wherein x₁, x₂, x₃, x₄ and x₅ may each be the same or different, and B₁, B₂, B₃, B₄ and B₅ may each be the same or different, and A may be the same or different from B₁, B₂, B₃, B₄ and B₅. For example, A may be a peptide or polypeptide that is highly expressed in a production host, such that the chimeric molecule facilitates increased production of the component molecules.

In accordance to still another one of the objects, there is provided a method as above, where the first component molecule is a peptide or protein or an active fragment thereof and at least one second component molecule is selected from the group consisting of: peptides, proteins, nucleic acids, carbohydrates, synthetic polymers, plant products, fungal products, small molecule drugs, detectable molecules, haptens, ligands, anti-infectives, and analogs and fragments thereof.

In accordance to one of the objects, there is provided a method as above where the chimeric molecule is a polyprotein.

In accordance to yet another one of the objects, there is provided a method as above where at least one of the component molecules is selected from the group consisting of: antigens, soluble receptors, growth factors, cytokines, lymphokines, chemokines, enzymes, anti-infectives, prodrugs, toxins, and active fragments thereof.

In accordance to still another one of the objects, there is provided a method as above where at least one of the component molecules is selected from the group consisting of: soluble p75TNFα receptor Fc fusion, human growth hormone, granulocyte colony stimulating factor (GCSF), granulocyte-macrophage colony stimulating factor (GM-CSF), interferon-α2b, pegylated (PEG) interferon-α, PEG-asparagase, PEG-adamase, anti-CO17-1A, hirudin, tissue type plasminogen activator, erythropoietin, human DNAase, IL-2, coagulation factor IX, IL-11, TNKase, activated protein C, PDGF, coagulation factor VIIa, insulin, interferon α-N3, interferon γ 1b, interferon α consensus sequence, platelet activating factor acetyl hydrolase and active fragments thereof.

In accordance to another one of the objects, there is provided a method as above where the first component molecule is a peptide, protein or an active fragment thereof and the second component molecule is a chemical compound. In an alternative embodiment, two or more or all of the component molecules are chemical compounds, such as, for example, hormones or carbohydrates or small molecules. In such instances, these chemical compounds are linked together with the present linker in vitro using conventional techniques. In accordance to another one of the objects, there is provided a method as above where at least one of the component molecules is an antibody. In one embodiment, the first component molecule is an antibody or an active fragment thereof and the second component molecule is other than an antibody. In another embodiment, second component molecule is an antibody or an active fragment thereof and the first component molecule is other than an antibody. In yet another embodiment, the first and second component molecules are each an antibody or an active fragment thereof.

In accordance to another one of the objects, there is provided a method as above, where at least one of the component molecules is selected from the group consisting of anti-microbial peptides, proteins, analogs, or active fragments thereof. In one embodiment, at least one of the component molecules is a defensin, a lysozyme, or a lactoferrin.

In accordance to another one of the objects of the present invention, there is further provided a method as above where at least one of the component molecules is selected from the group consisting of: peptides, proteins, analogs or active fragments thereof and they are human or non-human animal peptides, proteins, analogs or active fragments thereof. In another embodiment, they are plant peptides, proteins, analogs or active fragments thereof. In a further embodiment, they are fish or microbial peptides, proteins, analogs or active fragments thereof.

In accordance to yet another one of the objects, there is provided a method as above, where at least two of the component molecules are selected from the group consisting of: peptides, proteins, analogs or active fragments thereof.

In accordance to a further one of the objects, there is provided a peptide as above, where the peptide is selected from the group consisting of: IGF-I, EGF, PDGF, ITF, KGF, lactoferrin, lysozyme, fibrinogen, α₁-antitrypsin, erythropoictin, hGH, tPA, interferon alpha, interferon beta, interferon gamma, consensus interferon, insulin, human chorionic gonadotropin, diphtheria protein, and anti-hemophilic factor.

In accordance to another one of the objects, there is provided a method as above, where at least one of the component molecule is a hormone. In one embodiment, the hormone is selected from the group consisting of: estrogen, testosterone, and progesterone.

In accordance to a further one of the objects, there is provided a method as above, where at least one of the component molecules is selected from the group consisting of: a cytotoxic compounds such as taxol or its analogs or derivatives, enzyme inhibitors such as matrix metalloproteinase inhibitors, and anti-infectives.

In accordance to yet another one of the objects, there is provided a method as above, where at least two of the component molecules are selected from the group consisting: lactoferrin/lactoferrin; lactoferrin/lysozyme; lysozyme/lysozyme; lactoferrin/EGF; EGF/EGF; lactoferrin/ITF; ITF/ITF; ITF/EFG; EGF/KGF; KGF/KGF; ITF/KGF; KGF/PDGF; PDGF/PDGF; α₁-antitrypsin/MMP inhibitor; estrogen/progesterone; antibody/antibody and ITF/ITF, or analogs, variants, or derivatives thereof.

In accordance to one of the objects, there is provided a method as above, where the chimeric molecule is a vaccine. In one embodiment, the chimeric molecule comprises an adjuvant as one of the component molecules. In another embodiment, the vaccine comprises a component of a pathogenic organism. In as yet another embodiment, the vaccine is a cancer vaccine, and the component molecules are molecules that are over-expressed in a cancer cell

In accordance to one of the objects, there is provided a method as above, where the administration of the chimeric molecule achieves a biological effect selected from the group consisting of: diagnostic, prophylactic, therapeutic, and nutritional.

In accordance to another one of the objects, there is provided a method as above, where the chimeric molecule further comprises at least a fragment of an additional polypeptide, wherein the polypeptide is highly expressed in the production host.

In accordance to another one of the objects, there is provided a method as above, where the chimeric molecule further comprises a leader sequence for directing secretion of the chimeric molecule from the production host, such as, for example, a yeast host, a mammalian cell host or E. coli host, or for directing storage of the chimeric molecule in the production host, such as, for example, a plant host or a E. coli host.

In accordance to another one of the objects, there is provided a method as above, where the chimeric molecule comprises a targeting molecule. For example, the targeting molecule can direct the chimeric molecule to a specific site in the treated host for action.

In accordance to a further one of the objects, there is provided a method as above, where the chimeric molecule further comprises a purification tag, wherein the purification tag facilitates in vitro purification of the chimeric molecule after production from a production host.

In accordance to another one of the objects, there is provided a method as above, where the linker comprises two cleavage sites and a spacer between the cleavage sites.

In accordance to yet another one of the objects, there is provided a method as above, where the chimeric molecule is a component of an edible product. In one embodiment, the edible product is selected from the group consisting of: milk, a plant, a seed such as a cereal grain, a microbial cell, such as yeast or bacterium, for example, Lactobacillus, and derivatives and extracts thereof.

In accordance to another one of the objects, there is provided a method as above, where the chimeric molecule is administered orally, parenterally such as intravenously, subcutaneously, intraperitoneally, transdermally, intracardicly, or by inhalation.

In accordance to one of the objects, there is provided a method as above, where the chimeric molecule is not a nucleic acid molecule.

In accordance to one of the objects, there is provided a method as above, where at least one of the first or second component molecules is an antibody or an active fragment thereof and the antibody is selected from the group consisting of: anti-IL8, anti-CD11a, anti-ICAM-3, anti-CD80, anti-CD2, anti-CD3, anti-complement C5, anti-TNFα, anti-CD4, anti-α4β7, anti-CD40L (ligand), anti-VLA4, anti-CD64, anti-IL5, anti-IL4, anti-IgE, anti-CD23, anti-CD147, anti-CD25, anti-β2 integrin, anti-CD18, anti-TGFβ2, anti-Factor VII, anti-II_{b}IIₐ receptor, anti-PDGFβR, anti-F protein (from RSV), anti-gp120 (from HIV), anti-Hep B, anti-CMV, anti-CD14, anti-VEFG, anti-CA125 (ovarian cancer), anti-17-1 A (colorectal cell surface antigen), anti-anti-idiotypic GD3 epitope, anti-EGFR, anti-HER2/neu; anti-αVβ3 integrin, anti-CD52, anti-CD33, anti-CD20, anti-CD22, anti-HLA, and anti-HLA DR or an active fragment thereof.

In accordance to another one of the objects, there is provided a method as above, where the composition further comprises a pharmaceutically acceptable carrier or excipient.

In accordance to another one of the objects of the present invention, there is provided a kit that contains a composition comprising a chimeric molecule and a package insert, where the package insert comprises instructions for administration of composition to a human or non-human treated host, where the chimeric molecule comprises at least one first component molecule, at least one linker, and at least one second component molecule; where the linker comprises an enzyme cleavage site and where at least a first linker is operably linked to a first component molecule and a second component molecule to produce a non-naturally occurring linkage and cleavage site between the first component molecule and second component molecule; where the cleavage site is engineered for cleavage in vivo by a treated host enzyme and is resistant to cleavage in any production host; where, upon cleavage of the chimeric molecule at the cleavage site, at least one of the component molecules is functionally active; and where at least one of the first and second component molecules comprises one selected from the group consisting of a peptide, a protein, or an analog, an active fragment or derivative thereof.

In accordance to a further one of the objects, there is provided a kit as above, where the cleavage site in the chimeric molecule is engineered for cleavage by an enzyme in vivo, such as in the gastrointestinal tract of the treated host, and the enzyme is, for example, enterokinase.

In accordance to another one of the objects, there is provided a kit as above, where the cleavage site in the chimeric molecule is engineered for cleavage in vivo, extracellularly, either at a cell surface or at other than a cell surface.

In accordance to yet another one of the objects, there is provided a kit as above, where the cleavage site in the chimeric molecule is engineered for cleavage intracellularly in the treated host, such as by an endogenous host enzyme. In one embodiment, the chimeric molecule is not a combination of a protein transduction domain and a cytotoxic domain.

In accordance to still another one of the objects, there is provided a kit as above, where the cleavage site in the chimeric molecule is engineered for cleavage in vivo intracellularly in the treated host, and the cleavage site is not a viral pathogen activated cleavage site.

In accordance to another one of the objects, there is provided a kit as above, where the cleavage site is engineered for cleavage in vivo, intracellularly in the treated host, and the second component molecule is other than a cytotoxic molecule.

In accordance to still another one of the objects of the present invention, there is provided a chimeric molecule comprising a formula: A(xᵢBᵢ)ⁿ, wherein A represents the first component molecule, x represents the linker, B represents the second component molecule, i and n are each a positive integer, and where the chimeric molecule comprises at least one first component molecule, at least one linker, and at least one second component molecule; where the linker comprises an enzyme cleavage site and where at least a first linker is operably linked to a first component molecule and a second component molecule to produce a non-naturally occurring linkage and cleavage site between the first component molecule and second component molecule; where the cleavage site is engineered for cleavage in vivo by a host enzyme and is not susceptible to cleavage in a production host; where upon cleavage of the chimeric molecule at the cleavage site, at least one of the component molecules is functionally active; and where at least one of the first and second component molecules comprises one selected from the group consisting of a peptide, a protein, or an analog or an active fragment or derivative thereof.

In accordance to yet another one of the objects, there is provided a chimeric molecule as above, where the formula is selected from the group consisting of:

(a) A(x₁B₁);

(b) A(x₁B₁)(x₂B₂), where x₁ and x₂ may be the same or different, and B₁ and B₂ may be the same or different;

(c) A(x₁B₁)(x₂B₂)(x₃B₃), where x₁, x₂ and x₃ may each be the same or different, and B₁, B₂ and B₃ may each be the same or different;

(d) A(x₁B₁)(x₂B₂)(x₃B₃)(x₄B₄), where x₁, x₂, x₃ and x₄ may each be the same or different, and B₁, B₂, B₃ and B₄ may each be the same or different; and

(e) A(x₁B₁)(x₂B₂)(x₃B₃)(x₄B₄)(x₅B₅), where x₁, x₂, x₃, x₄ and x₅ may each be the same or different, and B₁, B₂, B₃, B₄ and B₅ may each be the same or different.

In accordance to another one of the objects, there is provided the chimeric molecule as above, where the chimeric molecule is a polyprotein.

In accordance to a further one of the objects, there is provided a nucleic acid molecule that encodes the chimeric molecule above. In a further aspect of the present invention, there is provided a vector that comprises the nucleic acid molecule encoding the chimeric molecule.

There is further provided a host cell comprising the nucleic acid molecule above.

In accordance to still another one of the objects of the present invention, there is provided a method for the preparation of a chimeric molecule in a production host for administration to a treated host comprising: (a) providing a nucleic acid that encodes a chimeric molecule; (b) transforming a production host with the nucleic acid; (c) allowing the production host to produce the chimeric molecule; (d) recovering the chimeric molecule from the production host; and (e) performing quality control on the harvested chimeric molecule to meet regulatory approval for administration to a treated host.

In accordance to another one of the objects, there is provided a method of preparation of a chimeric molecules as above, where the production host is selected from the group consisting of: a bacterial cell, including E. coli; a fungal cell, including yeast or Aspergillus; a plant cell; a plant seed, including a cereal grain, such as rice, wheat, rye, oats, and barley; a mammalian cell, such as CHO cells; an insect cell, such as SF9 cells; a plant, such as a tobacco plant; and an animal, such as transgenic cows, goats, sheep or pigs.

In accordance to yet another one of the objects, there is provided a composition comprising a chimeric molecule as above and a pharmaceutically acceptable carrier for administration to a treated host.

In accordance to another one of the objects, there is provided a composition as above, where the cleavage site is engineered for cleavage by a treated host enzyme in the gastrointestinal tract of the treated host, such as when the enzyme is enterokinase. In another embodiment, the cleavage site is engineered for cleavage at an inflammatory site, such as in the synovium, or at a tumor site, such as stomach cancer.

In accordance to another one of the objects, there is provided a method as above, where the composition is encapsulated.

In particular, the present invention is not any of the methods or compositions disclosed in the prior art, but may be improvements or modifications of such.

Other objects, features and advantages of the present invention will become apparent to a person of ordinary skill in the art upon reading the description herein. Such other objects, features and advantages are considered part of the present invention.

### BRIEF DESCRIPTION OF THE DRAWING

FIG. 1 is a diagrammatic representation of the types of enzymes (“target enzymes”) for which cleavage sites can be designed for the linkers of the chimeric molecules of the present invention.

### DETAILED DESCRIPTION OF THE INVENTION

The technical terms herein are to be understood as these terms are conventionally used in the art. The technical dictionaries that may be used in this regard includes: Lewin, Genes V, published by Oxford University Press, 1994 (ISBN 0-19-854287-9); Kendrew et al (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-8). Additionally, definitions of biotech terms may be accessed via websites such as: http://biotechterms.org. For a better understanding of the present invention, the following terms shall have the following particular meaning:

The term “active fragment” or “biologically active fragment” means a portion of a molecule, such as a protein, a nucleic acid molecule, or an antibody, having biological activity or having the ability to participate in such activity, including but not limited to: the ability to bind to another molecule specifically, such as in an antibody/antigen reaction or a DNA/DNA or DNA/RNA hybridization, such as for diagnostic purposes, the ability to act as an antigen or immunogen, having enzymatic activity, having an enzyme recognition site, being able to act as an enzyme substrate, ability to interact with a ligand or a receptor, and ability to inhibit other biologically active molecules. Such fragments may exhibit an activity that is similar, but not necessarily identical, to an activity of a naturally occurring nucleic acid, polypeptide, or antibody. The biological activity of the fragments herein includes an improved desired activity or a decreased undesirable activity. An example of an active fragment of an antibody, for example, is the F_{c} or F_{ab} fragment of an immunoglobulin, or the variable region of a heavy chain, or the variable region of a light chain of the immunoglobulin.

The term “analogs” means molecules that have at least about 70% sequence homology to the molecules being compared. The differences between a molecule and its corresponding analog may include, for example, but are not limited to: conservative amino acid changes or its corresponding codon changes; deletion of one or more amino acid residues or its corresponding codon, for example, to eliminate one or more disulfide linkage sites; addition of an amino acid or its codon such as methionine, for example, to aid in bacterial expression; conservative changes in the side chain of a chemical molecule that does not affect the binding of the chemical molecule, for example, change from a methyl group to an ethyl, propyl or butyl group; or such other similar examples.

The term “antibody” refers to an antibody naturally found or induced in humans or non-human animals, a polyclonal antibody, a monoclonal antibody, a humanized antibody, a single chain antibody, as well as to fragments thereof, such as F_{ab} or F_{c} fragments or variable regions of the light or heavy chain of an immunoglobulin.

The term “anti-infectives” includes antibacterial, antiviral, antifingal and other anti-pathogen molecules or compounds, that have either cytostatic or cytocidal activities, that act either directly or indirectly by inducing the production of molecules that have a direct cytostatic or cytocidal effect. An example of an anti-infective is a defensin, but is not limited to such.

The term “binds specifically” in the context of antibody binding, refers to high avidity and/or high affinity binding of an antibody to a specific polypeptide or, more accurately, to a specific epitope of a specific polypeptide. Antibody binding to such specific epitope is typically stronger than binding of the same antibody to any other epitope or any other polypeptide that does not contain such specific epitope. Such specific antibodies are typically produced by injecting the specific polypeptide into an animal to elicit the production of such antibodies. Such a specific antibody may be capable of binding other polypeptides at a weak, yet detectable level (for example, 10% or less of the binding shown to the specific polypeptide). Such weak binding is readily discernible from the specific antibody binding, for example, by use of appropriate controls. In general, antibodies of the invention specifically bind to a specific polypeptide with a binding affinity of 10⁻⁷ M or more, preferably, 10⁻⁸ M or more (for example, 10⁻⁹ M, 10⁻¹⁰ M, 10⁻¹¹ M, and the like).

The term “biological activity” in reference to a molecule includes: the ability of the molecule to be detected, thus, a diagnostic activity; the ability of a molecule to act as a vaccine or adjuvant, thus, a prophylactic activity; the ability of a molecule to act as a therapeutic for treating a disease or condition, thus, a therapeutic activity; the ability of the molecule to inhibit growth and proliferation of microorganisms, such as bacteria, viruses, fungi, prions, parasites, etc., thus, an anti-infective activity; the ability of a molecule to enhance nutritional value of food, thus, a nutritional activity; and the ability of the molecule to participate in other biological reactions, such as: enzymatic reactions; binding activities such as in immunological, antibody-antigen binding, ligand-receptor binding or in signal transduction reactions and such similar activities.

The term “biological effect” refers to the results of any biological activity including, for example, a diagnostic effect, a prophylactic effect, a therapeutic effect, an anti-infective effect, a nutritional and other biological effects as conventionally understood.

The term “endogenous treated host molecule” or “endogenous treated host enzyme” refers to a molecule or enzyme that is encoded by the genome of the treated host.

An “expression cassette” is a nucleic acid construct generated recombinantly or synthetically, that contains a series of specified nucleic acid elements that can be transcribed or translated to produce one or more recombinant polypeptides in a host expression system. The expression cassette can be incorporated into a plasmid or a viral vector, for example, to form an expression vector, or can be integrated into host chromosome, mitochondrial DNA, plastid DNA, virus, or nucleic acid fragment, for example, by particle bombardment. Typically, the expression cassette includes, among other sequences, a promoter, a transcription start, a translation start, a heterologous gene of interest, a translation terminator and a transcription terminator. Optionally, the expression cassette may contain one or more selectable markers.

An “expression vector” refers to a vector that contains or is suitable for use with an expression cassette for expression of heterologous DNA or RNA in a host cell. Many prokaryotic and eukaryotic expression vectors are commercially available. Selection of appropriate expression vectors is within the knowledge of those having skill in the art. Optionally, an expression vector may contain one or more selectable markers for selection of host cells that contain the expression vector.

The term “extracellular” as it relates to cleavage of the chimeric molecule of the present invention refers to cleavage of the chimeric molecule outside of a cell of the treated host, such as, for example, in the gastrointestinal tract, in blood, in lymphatic fluid, peritoneal fluid, interstitial fluid, spinal fluid, synovial fluid, vaginal fluid or lung fluid and such similar space.

The term “intracellular” as it relates to cleavage of the chimeric molecule of the present invention refers to cleavage of the chimeric molecule inside a cell in a treated host.

The term “microbial cell” in reference to an edible product includes micro-organisms such as yeast and Lactobacillus, that are approved for human or animal consumption.

The term “microbial proteins” means proteins that are derived from or are substantially identical to those proteins obtainable from microorganisms, including but not limited to: bacteria, viruses, fingi, prions, other single cell organisms, parasites, and analogs of such.

The term “molecule” include any compound or salts thereof, whether naturally occurring or synthetically made, and includes a peptide, an oligopeptide, a polypeptide, a protein including a glycoprotein, a nucleic acid, whether DNA or RNA, a carbohydrate, a natural product such as a plant product, other polymers including synthetic polymers and fragments, a hormone, a chemical compound such as taxol, its analog or derivative, combinations and analogs thereof.

The term “naturally occurring” refers to any molecule existing in nature in a form that is not the result of intervention of the hand of man.

The term “operably linked” as used in reference to the linkage between the component molecules and the cleavage site in the chimeric molecule means that component molecules are linked in such manner that, for example, upon cleavage of the chimeric molecule at the cleavage site, the component molecules are capable of exhibiting one or more of its biological activities.

The term “pharmaceutically acceptable carrier” as used herein means a carrier that is appropriate for the mode of delivery of the chimeric molecule or composition containing the chimeric molecule. For example, for parenteral administration, an acceptable carrier can be saline; for oral administration, an acceptable carrier may be a food product that is genetically engineered to contain the chimeric molecule such as rice, milk, vegetables and the like, where the food product may have been processed or extracted. A pharmaceutically acceptable carrier is generally a non-toxic solid, semisolid or liquid filler, diluent, encapsulating material or formulation auxiliary of any conventional type. It is non-toxic to recipients at the dosages and concentrations employed and is compatible with other ingredients of the formulation. For example, the carrier for a formulation containing polypeptides preferably does not contain oxidizing agents and other compounds that are known to be deleterious to the half-life or shelf-live of the polypeptides. Suitable carriers include, but are not limited to: water, dextrose, glycerol, saline, ethanol, and combinations thereof. The carrier may contain additional agents such as wetting or emulsifying agents, pH buffering agents, or adjuvants which enhance the effectiveness of the formulation. Other materials such as anti-oxidants, humectants, viscosity stabilizers, and similar agents may be added as necessary. Percutaneous penetration enhancers such as Azone may also be included. Compositions for oral administration herein may form solutions, suspensions, tablets, pills, capsules, sustained release formulations or powders.

The term “pharmaceutically acceptable salts” suitable for use herein include the acid addition salts (formed with the free amino groups of the polypeptide) and those that are formed with inorganic acids such as, for example, hydrochloric or phosphoric acids, or such organic acids as acetic, mandelic, oxalic, and tartaric. Salts formed with the free carboxyl groups may also be derived from inorganic bases such as, for example, sodium, potassium, ammonium, calcium, or ferric hydroxides, and such organic bases as isopropylamine, trimethylamine, and the like.

The term “plant” in reference to an edible product includes vegetables and grains, such as cereal grains, typically, rice, wheat, barley, corn, millet, sorghum and oats, for example.

The term “polypeptide” is a “molecule” that is a polymer of amino acids that may or may not be additionally post-translationally modified by the “production host,” such as via glycosylation, or modified in vitro, such as by chemical addition of synthetic polymers, including polyethylene glycol. The term “polypeptides” and “polypeptide compositions” are used to refer to peptides, oligopeptides, proteins, analogs, and active fragments or derivatives thereof.

The term “production host” refers to the host system for producing the chimeric molecules of the present invention. Such a host system includes host cells, either in vitro or in vivo, that can be or has been the recipient of any recombinant vector or vectors, plasmids, or isolated polynucleotides encoding the chimeric molecules and the progeny thereof.

The term “protein” may be synonymous with the term “polypeptide” or may refer, in addition, to a complex of two or more polypeptides and may be in primary, secondary or tertiary configuration.

The term “target enzyme” refers to the enzyme for which the cleavage site of the chimeric molecule of the present invention is designed. For example, if the chimeric molecule herein is designed with a cleavage site for enterokinase, “enterokinase” is the target enzyme.

The term “treated host” refers to the host to which delivery of the chimeric molecule of the present invention is intended so as to produce a biological effect including a diagnostic, prophylactic, therapeutic or nutritional effect. Such treated hosts include, but is not limited to: humans, non-human animals such as farm animals including cattle, pigs, goats and horses, and domestic animals such as dogs and cats; as well as rodents; non-human primates; birds such as chickens; plants; microorganisms; parasites; and fish. A “treated host” may include two hosts as, for example, where a chimeric molecule containing a cleavage site specific to a microorganism (hereafter, a “targeted microorganism”) is administered to a subject and the microorganism transits through in the GI tract of the subject. The chimeric molecule may be cleaved intracellularly by the targeted microorganism or released intact by the targeted microorganism for cleavage by the “treated host” enzyme, that is, an enzyme of the subject. For example, if the chimeric molecule carries a detectable signal, such as green fluorescent protein, for example, that is activated upon cleavage, presence of the green fluorescent protein will indicate presence of the microorganism in the gut of a human. The terms “individual,” “subject,” “patient,” and “treated host” are used interchangeably herein.

Before the present invention is further described, it is to be understood that the invention is not limited to the particular embodiments described, as such may, of course, vary. It is to be understood that the terminology used herein is for the purpose of describing particular embodiments only, and is not intended to be limiting, since the scope of the present invention will be limited only by the appended claims.

Where a range of values is provided it is understood that each intervening value to a tenth of the unit of the lower limit, unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

All publications mentioned herein, including patents, patent applications, and published literature are incorporated herein by reference in their entireties including publications cited therein to disclose and describe the methods and/or materials in connection with which the publications are cited.

It must be noted that as used herein and in the appended claims, the singular forms of a term, such as “a,” “an,” “the,” “polypeptide,” “polynucleotide,” “chimeric molecule,” and “molecule” include the corresponding plural forms unless the context clearly indicates or dictates otherwise. For example, reference to “a polypeptide” includes a plurality of polypeptides and reference to “an agent” includes one or more agents.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

The invention described below is given by way of example, and is not to be interpreted in any way as limiting the invention.

The inventor herein has discovered that two or more component molecules can be advantageously combined to form a non-naturally-occurring chimeric molecule, by use of one or more linkers that contain one or more cleavage sites, for administration to a host (that is, a “treated host”), where the component molecules can be released by cleavage molecules, such as enzymes, present in the treated host. The chimeric molecules herein are designed in such a manner as to be cleavable into component parts, preferably, at a desired location in the treated host to achieve a biological effect either at the site of cleavage or at a location close by. Cleavage of the chimeric molecules may take place in a substantially confined area in the treated host, such as in the gastrointestinal tract (“GI”), in synovial fluid, or inside a cell, for example, or cleavage may take place systemically, such as in the blood or other body fluids. Cleavage of the chimeric molecules releases component molecules that are functional in the treated host. Such component molecules may or may not be active prior to cleavage from the chimeric molecule. In one embodiment of the present invention, at least one of the component molecules in the chimeric molecule is a peptide, a polypeptide or an active fragment thereof.

Thus, the present invention includes methods of delivering component molecules to a treated host to achieve a biological effect therein by administering chimeric molecules thereto, each chimeric molecule containing at least two component molecules, each of which are linked to another by a linker that contains one or more cleavage sites for cleavage by cleavage molecules in the treated host. The present invention includes chimeric molecules, nucleic acid molecules encoding such, vectors and host cells containing such nucleic acid molecules, kits and compositions containing the chimeric molecules or the encoding nucleic acid molecules, and methods of making and using the same. In particular, the chimeric molecules of the present invention are non-naturally occurring. [0125] In its simplest configuration, the chimeric molecules of the present invention has a formula: “AxB,” where “A” is a first component molecule, “B” is a second component molecule and “x” is a linker that contains one or more cleavage sites. However, the chimeric molecule of the present invention is not limited to “AxB” but includes chimeric molecules having a formula: A(x₁B₁)ⁿ, where “i” and “n” are each positive integers and “xB” is primarily a unit that can be repeated (hereafter, a “repeat”). Thus, for example, the present chimeric molecule includes chimeric molecules having the formulas: (Ax₁B₁) or (Ax₁B₁)(x₂B₂). Optionally, the chimeric molecule can have a formula of (Ax₁B₁)(x₂B₂)(x₃B₃), (Ax₁B₁)(x₂B₂)(x₃B₃)(x₄B₄), or (Ax₁B₁)(x₂B₂)(x₃B₃)(x₄B₄)(x₅B₅), and so on, including any number of repeats of (xB) units that can be reasonably produced and administered, where each “B” can be the same or different, and can further be the same or different from “A”; and each “x” can be the same or different. In some embodiments, the component molecule B that forms a repeat is small. For example, where the molecule is a small peptide which, if administered alone, would be quickly degraded; for example, an anti-infective peptide such as defensins or the intestinal trefoil factor (“ITF”). Further, it is not necessary for all the cleavage sites in the chimeric molecules to be cleaved at the same time or completely. One or more component molecules may be cleaved from the chimeric molecule while other component molecules remain as part of the remaining chimeric molecule. As an example, the chimeric molecule herein may bind to a tissue, such as an extracellular matrix, in an uncleaved or partially cleaved form, and component molecules may be released therefrom from time to time when a certain enzyme level at that location is high. In addition, the component molecules may be active as part of the chimeric molecule without being cleaved as long as the active site of such component molecule is free to interact with other molecules.

The present invention includes chimeric molecules that have cleavage sites that are designed for cleavage at a desired location in the treated host. For example, the chimeric molecule herein may be designed for cleaved by an enzyme in the GI tract of the treated host to release component molecules for activities therein, such as anti-infective activity. An application of this embodiment is providing animal or chicken feeds containing the present chimeric molecules to provide for anti-infective activities without the use of antibiotics. This application is useful for humans as well, especially in the case of baby foods, such as in milk, milk products, fruits, cereals, meats, and juices. In such an instance, the chimeric molecule is constructed with a linker that has one or more cleavage sites for one or more enzymes in the GI tract, such as an enterokinase cleavage site, for example. The amino acid sequence representing the enterokinase recognition or cleavage site is known and is generally represented by the amino acid sequence: -Lys-Lys-Lys-Lys-Asp-. The chimeric molecule with an enterokinase cleavage site can be made in any conventional manner using recombinant techniques in any number of suitable host expression systems (“production host”). One example of such is described in U.S. Pat. No. 4,769,326, entitled “Expression Linkers,” or its corresponding European counterpart EP0035384. Besides enterokinase, for cleavage of the chimeric molecules in the GI tract, linkers containing cleavage sites for other GI tract enzymes can be used.

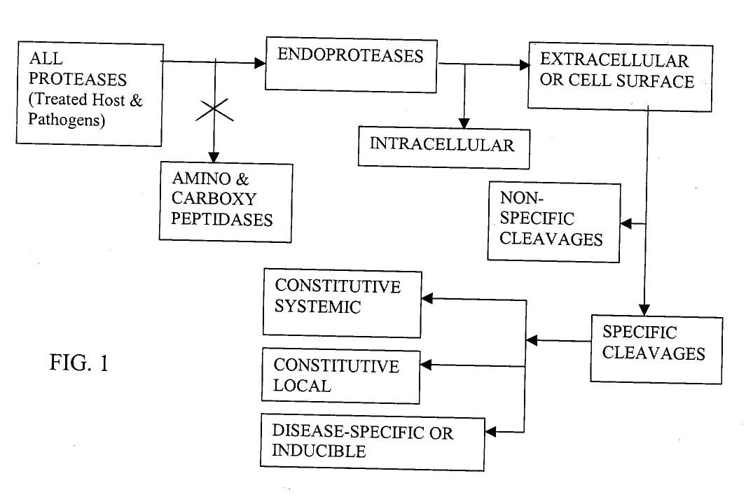

The types of cleavage sites suitable for incorporation into the linkers of the present chimeric molecules include certain ones that can be cleaved by certain treated host enzymes (hereafter, “target enzymes”), as illustrated in FIG. 1. Starting with all proteases present in a treated host, including those endogenous to the treated host and those that may be introduced by infecting pathogens, the cleavage sites suitable for use herein exclude those that are substrates for amino and carboxy peptidases and exclude those that are non-specific. However, less specific endopeptidases, such as trypsins, chymotrypsins, and elastases, will find use herein. In one embodiment of the present invention, the cleavage sites include those that are substrates for endopeptidases. In an aspect of this invention, the cleavage sites suitable herein include those that are substrates for intracellular enzymes. In another aspect of the present invention, the cleavage sites include those that are substrates for extracellular enzymes. In a further aspect of the present invention, the cleavage sites include those that are substrates for enzymes that are active at a cell surface. Notably, the target enzymes are constitutively expressed or are inducible. They circulate either systemically or locally.

The present invention further includes chimeric molecules having cleavage sites that are designed for intracellular cleavage in the treated host. In one aspect of the invention, the cleavage site is designed for cleavage by an intracellular enzyme that is endogenous to the treated host. In another aspect of the invention, the cleavage site is designed for cleavage by any enzyme present intracellularly in the treated host, whether endogenous or not, provided that the chimeric molecule is not a combination consisting of a transduction domain and a cytotoxic domain or that the second component molecule is not a cytotoxic molecule. In another aspect of the invention, the cleavage site is designed or engineered for cleavage intracellularly in the treated host, provided that the cleavage site is not a pathogen activated cleavage site from a pathogen infecting the treated host cell. Thus, for example the cleavage site of the present invention may be designed for an enzyme to be separately induced in or introduced into the treated host.

The present invention also includes administration of chimeric molecules having a structure as above but with cleavage sites that are designed for enzymatic cleavage extracellularly in the treated host, regardless of whether the enzyme is endogenous to the host or not, constitutively expressed in the host or inducible in the host. Extracellular cleavage can take place anywhere in the host, such as, for example, in any body fluids, including but not limited to: lymph fluids, blood, synovial fluids, peritoneal fluids, spinal fluids, vaginal secretions and lung fluids. Extracellular cleavage can be cleavage on the surface of a cell. The present invention thus includes chimeric molecules containing linkers with cleavage sites designed for enzymatic cleavage at a cell surface in a treated host.

In light of the present invention, the selection of appropriate enzyme cleavage sites and sequences therefor, for use in the chimeric molecules herein for cleavage at a desired location inside a treated host is within the skill of a person in the art. Information regarding enzymes and their cleavage sites are available from numerous sources. For example, the website at http://us.expasy.or/cgi-bin/enzyme-search-cl displays a list of definitions of enzyme classes. As an illustration, class “3. 4. -.-” are enzymes “Acting on peptide bonds (peptide hydrolases).” Class “3. 4.24.-” are “Metalloendopeptidases.” Further, for example, clicking the link to “3.4.21.9 Enteropeptidase” brings up the next page giving more information on this enzyme. It provides “enterokinase” as the alternative name. It specifies “Selective cleavage of -Lys-/-Ile bond in trypsinogen” as being the reaction catalyzed. It provides references to articles in Medline relating to this enzyme. In another example, clicking the link to “3.4.21.38 Coagulation factor XIIa” brings up the next page listing Hageman factor as the alternative name to Coagulation factor XIIa, and stating “Cleaves selective Arg-/-Ile bonds in factor VII to form factor VIIa and factor XI to form factor XIa,” as the reaction catalyzed.

Another reference source for enzymes and their cleavage site is AHFS Drug Information, published annually by the American Society of Health-System Pharmacists, Inc. (7272 Wisconsin Avenue, Bethesda, Md. 20814, USA). For example, under “20:40 Thrombolytic Agents p. 1477,” Alteplase is listed as a thrombolytic agent and is “a biosynthetic (recombinant DNA origin) form of the enzyme human tissue-type plasminogen activator (t-PA).” Further, “[e]ndogenous human t-PA is secreted as a one-chain polypeptide, which may be cleaved at the arginine²⁷⁵-isoleucine²⁷⁶ peptide bond by several endogenous proteases, including plasmin, tissue kallikrein, activated factor X (factor Xa), and trypsin, to form a two-chain derivative.”

Moreover, published literature, for example, those available through the government website: http://www.ncbi.nlm.nih.gov/entrez/query.fcgi, is another source of information on enzymes and cleavage sites for use in the chimeric molecules of the present invention. For example, by entering “arthritis” and “protease” as search terms, over 2000 articles on the subject matter can be found. One can quickly discern that matrix metalloproteinase 3 (“MMP-3”, also known as “stromelysin-1”) is strongly expressed in normal and early degenerative stages of osteoarthritis, and MMP-2 and MMP-11 are up-regulated in late-stage disease, as described in Aigner, T. et al., Arthritis Rheum. 44(12): 2777-89 (December 2001). Moreover, cathepsin K, having potent aggrecan-degrading activity, has also been found to be highly expressed in synovial fibroblasts, and cathepsin K-generated aggrecan cleavage products were found to specifically potentiate the collagenolytic activity of cathepsin K, as described in Hou W. S. et al., Am. J. Pathol. 159(6): 2167-77 (December 2001). Thus, identification of the appropriate treated host enzyme cleavage site for incorporation into the chimeric molecules of the present invention is within the skill of a person in the art.

As a further example, Tortorella, M. D. et al., J. Biol. Chem. 275(24): 18566-18573 (Jun. 16, 2000), discloses the cleavage of aggrecan, a major proteoglycan of cartilage that is the first matrix component to undergo measurable loss in arthritic diseases, by recombinant human aggrecanase-1 (“ADAMTS-4”). Aggrecanase-1 and aggrecanase-2 (“ADAMTS-11/5”) are members of the adamalysin family of zinc-binding metalloproteases. Tortorella et al. reported that the recombinant human aggrecanase-1 cleaved aggrecan at several sites, all sites containing a glutamic acid residue in the P₁ position and a non-polar or uncharged polar residue (alanine, leucine, or glycine) in the P₁′ position. The most efficiently cleaved site was Glu¹⁶⁶⁷-Gly¹⁶⁶⁸ bond in the G2-G3 domain of the molecule. The GI fragment of the molecule was further cleaved at Glu¹⁴⁸⁰-Gly¹⁴⁸¹, and cleavage at Glu³⁷³-Ala³⁷⁴ occurred more slowly. Further, the authors reported that aggrecanase-1 and aggrecanase-2 did not cleave at a Asn³⁴¹-Phe342 site, making it the only enzymes to-date that have been shown to cleave at the aggrecanase Glu³⁷³-Ala³⁷⁴ site without also cleaving at the matrix metalloproteinase (“MMP”) site. The authors further reported that other studies have shown that two proteases, MMP-8 and atrolysin-C, that cleaved at the aggrecanase Glu³⁷³-Ala³⁷⁴ site, also cleaved at the Asn 34-Phe342 MMP site. Hence, in the design of a linker for delivery of component molecules to the synovial fluid for treatment of arthritis, for example, a Glu-Ala sequence can be incorporated into the present linker, with a glutamic acid residue in the P₁ position and a non-polar or uncharged polar residue (alanine, leucine, or glycine) in the P₁′ position; or a Glu-Gly sequence may be used.

Examples of target enzymes for which cleavage sites may be included in the chimeric molecules of the present invention are many and will be known to a person skilled in the art. Some examples are shown in Table 1 and include, but are not limited to: enterokinase (active in the gut); coagulation factors such as Factors VIIa, IXa, Xa, XIa, and XIIa (active in blood); ADAMTS-4, -5 (aggrecanase-1, -2) (active in joints, heart, brain, lung); thrombin (active in blood); plasmin (active in blood); complement factors such as Factor D, C1r, C3/C5 convertase (active in blood); gastrin (active in stomach); granule proteases such as elastase and PR-3 (active in neutrophils and leukocytes and secreted as active forms); matrix metalloproteinases (“MMPs” most of which are secreted as zymogens) such as MMP-2 (upregulated in breast and prostate cancer and in injured liver), MMP-7 (Matrilysin, active in glandular epithelium such as colon, and upregulated in tumors), MMP-9, MMP-11, MMP-13 (MMP-11 and MMP-13 are up-regulated in breast cancer); membrane type MMPs such as MT-1, MT-2, MT-3 and MMP-14, MMP-15, MMP-16, MMP-24 (transmembrane proteins that are active at cell-surface; some are shed, upregulated in metastases); type II transmembrane serine proteases such as TMPRSS-2, -4 and Matriptase (transmembrane proteins that are active at cell surfaces, upregulated in tumors); ADAMs family of about 30 disintegrin and metalloproteinases including ADAM-10, ADAM-17 and TACE (TNF convertase) (expressed in most tissues and are active at plasma membrane); neprilysin (expressed in normal and neoplastic liver cells; active at plasma membrane), cathepsin K (secreted by synovial fibroblasts), mast cell tryptase (activated in asthma) and tissue type plasminogen activator.

In some embodiments, the cleavage sites of the chimeric molecules of the present invention includes not only those that are substrates for proteases, but includes those that are substrates for other enzymes, such as glycosidases and heparanases.

In another embodiment, the enzyme cleavage site or sites engineered into the chimeric molecule are designed for enzymes that are expressed or heightened under disease, stress, pathogenic, allergic, premature birth or geriatric conditions, and other conditions requiring treatment.

The linker of the present invention includes those having one or more than one enzyme cleavage sites. The linkers herein can advantageously include a spacer molecule for example, so as to better expose the cleavage site to enzymes for cleavage. Thus, in one embodiment, the present invention includes a spacer in the linker to better expose the cleavage site to enzymatic action. In such instances, the linker can be a series of random amino acid residues that do not tend to fold upon themselves. These amino acid residues can thus be a chain of hydrophilic amino acid molecules, for example. Further, when a spacer is used, the present invention may optionally include the addition of another cleavage site in the linker such that the spacer may be cleaved together with the cleavage site to generate component molecules having appropriate or natural C-terminals or N-terminals or the appropriate active fragments.

In one aspect of the present invention, where component molecules on each side of the linker are active prior to cleavage and for good protease accessibility, the linker herein optionally contains about 10 to 20 amino acid residues, more preferably about 11-17 amino acid residues (hereafter, a “spacer”). For example, the chimeric molecule of the present invention may contain a spacer between Protein X and Protein Y, where the spacer contains amino acid sequences such as: Protein X-ASGGGGIEGRGGGGSA-Protein Y, where the sequence in bold and underlined represents a Factor Va cleavage site and proteins X and Y are component molecules. Thus, additional amino acids may be engineered into the chimeric molecule upstream and/or downstream of the enzyme cleavage site to ensure exposure of the cleavage site to the cleaving enzyme while maintaining component molecule activity. Examples of such amino acids are known in the art, such as, for example, see Hosfield, T. and Lu, Q., “Influence of the Amino Acid Residue Downstream of (Asp)₄Lys on Enterokinase Cleavage of a Fusion Protein,” Anal. Biochem. 269: 10-16 (1999).

In another aspect of the present invention, for example, where the component molecules are intended not to be active until cleaved, fewer amino acid residues can be used. For example, the chimeric molecule may have an amino acid sequence: Protein X-GGRSGG-Protein Y, where RS represents cleavage site for plasmin and proteins X and Y are the component molecules:

The present invention includes an embodiment where in rare instances, the fused component molecules, when joining the C terminus of one component molecule to the N terminus of a second component molecule may itself create a cleavage site upon cleavage without addition of a linker molecule.

In one embodiment of the present invention, when the cleavage site is designed for extracellular cleavage, other than at a cell surface, the chimeric molecule is other than glycosylated interferon beta.

The component molecules herein can be any molecules that can be expected to achieve a biological effect in the host. Thus, the present invention include component molecules that are peptides, proteins, nucleic acids, carbohydrates, other natural or synthetic polymers, small molecule drugs, detectable molecules such as for diagnostic purposes, haptens, ligands, anti-infectives, and analogs and active fragments thereof.

The component molecules herein also can be of any origin or source, human or non-human, natural or synthetic. Thus, for example, the component molecules can be peptides, proteins, analogs or derivatives thereof that are substantially identical to those obtainable from human, non-human animals, plants, fish, insects, and microbes including bacteria, viruses, fungi and parasites.

In another aspect of the present invention, the chimeric molecule is a polyprotein, where at least two or all of the component molecules are peptides or polypeptides or active fragments thereof (hereafter, “protein components”). The protein components that are suitable for use herein include, but are not limited to: antibodies, antigens, receptors, growth factors, hormones, cytokines, lymphokines, chemokines, enzymes, anti-infectives, prodrugs, toxins, nutrition-enhancing molecules, and active fragments thereof.

TABLE 1
Sample Target Enzymes: Endoproteases useful for cleavage of chimeric
molecules in vivo in a treated host.

| | Cleavage Site | | |
|---|---|---|---|
| | ( . . . P1 * P1′. . . ) | Location | Expression/Use |
| Enterokinase | DDDDK*A | | Duodenum and intestine |
| Coagulation Factors: | | | |
| Factor Xa | IGER * T (P1′ not R/T) | | Blood |
| Factors VIIa, IXa, XIIa | R * I | | |
| Factor XIa | R * AIV | | |
| ADAMTS 4,5 | KEEE * GLSS | | Joints, heart, brain, lung |
| Aggreganases 1,2 | | | (for arthritis) |
| Thrombin | (P4)(P3)PR * (P1′)(P2′) | | Blood |
| | where | | |
| | (P3)(P4) = hydrophobic | | |
| | (P1′)(P2′) = non-acidic | | |
| Plasmin | K/R * S | | Blood |
| Complement Factors: | | | |
| Factor D | R * K | | Blood |
| Clr | K/R * I | | |
| C3/C5 Convertase | R * S | | |
| Gastricin | Y * (P1′) | | Gastric juice |
| Granule Proteases: | | | |
| Elastase | AAPV * (P1′) | Secreted as active forms | Neutrophils, leukocytes |
| PR-3 | PLAQAV * RSSS | | |
| Matrix | | | |
| Metalloproteinases: | | | |
| MMP-7 (Matrilysin) | ELR * EST | Most secreted as | MMP-7 in glandular |
| Also MMPs | | zymogens | epithelium (colon) but |
| 2, 9, 11, 13, etc | | | up in tumors, as are |
| | | | others: MMP 2, 11, 13 in |
| | | | breast cancer. MMP-2 |
| | | | also up in prostate |
| | | | cancer and liver injury |
| Membrane-Type MMPs: | | | |
| MT 1, 2, 3; MMPs 14- | | Cell-surface, | Up in metastasis |
| 16; 24 | | transmembrane proteins; | |
| | | some shed | |
| Type II Transmembrane | | | |
| Seine Proteases: | | | |
| TMPRSS 2, 4; | Trypsin-like | Cell-surface, | up in tumors |
| Matriptase | | transmembrane proteins | |
| ADAMs: | | | |
| A disintegrin and | PLAQA * VRSS | Plasma membrane | Most tissues |
| metalloproteinase | | | |
| family: about 30 | | | |
| members, incl. ADAM | | | |
| 10, 17 | | | |
| TACE (TNF convertase) | | | |
| Neprilysin | (P1) * F/Y | Plasma membrane | Normal and neoplastic |
| | where (P1) = | | liver |
| | hydrophobic | | |
| Caspases: | | | |
| 1, 3, 8, 9 | Selective, overlapping | Cytoplasmic | Ubiquitous, inducible by |
| Capase 3 | DEVD * G | | cell stress |
| | | | Casp 1 activates IL-18 |

In a further embodiment of the present invention, at least one of the component molecules is selected from the group consisting of: soluble p75TNFα receptor Fc fusion, human growth hormone, granulocyte colony stimulating factor (“GCSF”), granulocyte-macrophage colony stimulating factor (“GM-CSF”), interferon-α2b, pegylated (“PEG”) interferon-α, PEG-asparagase, PEG-adamase, anti-CO17-1A, hirudin, tissue type plasminogen activator, erythropoietin, human DNAase, IL-2, coagulation factor IX, IL-11, TNKase, activated protein C, PDGF, coagulation factor VIIa, insulin, interferon α-N3, interferon γ 1b, interferon α consensus sequence, platelet activating factor acetyl hydrolase and active fragments or derivatives thereof.

In another embodiment of the present invention, the chimeric molecule contains as component molecules peptides, proteins or active fragments thereof that are selected from the group consisting of: interleukins; growth factors including IGF-I, EGF, FGF, PDGF, ITF, and KGF; colony stimulating factors including GM-CSF and M-CSF; coagulation factors including Factor VIII or Factor IX, tPA; growth hormones including hGH; anti-infectives including lactoferrin and lysozyme; fibrinogen; α₁-antitrypsin; erythropoietin; interferons including interferon alpha, interferon beta, interferon gamma, and consensus interferon; insulin; human chorionic gonadotropin; diphtheria protein; anti-hemophilic factor; receptors; vaccines; antibiotics; or analogs or fragments thereof.

In a preferred embodiment of the invention, it is particularly desirable to use as component molecules, those drugs that have been approved by the Food and Drug Administration (“FDA”), listings of which can be found at the website: www.FDA.gov. For example, for biological molecules that have been approved under Biologics, the link under 2002 Biological License Application Approvals lists molecules such as interferon beta-1 a (tradename “Rebif”), for treating relapsing forms of multiple sclerosis; diphtheria and tetanus toxoids and acellular pertussis vaccine (tradename, “Daptacel”); peginterferon α-2a (tradename “Pegasys”) for treatment of adults with chronic hepatitis C; and adalimumab (tradename “Humira”), which is a recombinant human IgG1 monoclonal antibody specific for human tumor necrosis factor (“TNF”) for rheumatoid arthritis. Other approved biologics are listed under the year of approval.

In one embodiment of the present invention, none of the component molecules are antibodies.

In another embodiment of the present invention, one or two or more of the component molecules are antibodies or active fragments thereof (hereafter, “antibody components”). The antibody components herein include any that are suitable for therapeutic, prophylactic or diagnostic purposes. In a preferred embodiment, the antibody components are selected from a list of antibodies that have been approved by the FDA. Examples of such antibodies include, but are not limited to: anti-IL8, anti-CD11a, anti-ICAM-3, anti-CD80, anti-CD2, anti-CD3, anti-complement C5, anti-TNFα, anti-CD4, anti-α4β7, anti-CD40L (ligand), anti-VLA4, anti-CD64, anti-IL5, anti-IL4, anti-IgE, anti-CD23, anti-CD147, anti-CD25, anti-β2 integrin, anti-CD18, anti-TGFβ2, anti-Factor VII, anti-II_{b}IIₐ receptor, anti-PDGFβR, anti-F protein (from RSV), anti-gp120 (from HIV), anti-Hep B, anti-CMV, anti-CD14, anti-VEFG, anti-CA125 (ovarian cancer), anti-17-1 A (colorectal cell surface antigen), anti-anti-idiotypic GD3 epitope, anti-EGFR, anti-HER2/neu; anti-αVβ3 integrin, anti-CD52, anti-CD33, anti-CD20, anti-CD22, anti-HLA, anti-TNF, and anti-HLA DR.

In one embodiment, the first component molecule is an antibody or an active fragment thereof and the second or other components are not antibodies or antibody fragments. In a further embodiment, the first component molecule is not an antibody or antibody fragment but the second or other component molecules are antibodies or their fragments. In a variation of the invention, all the component molecules of the chimeric molecule are antibodies or active fragments thereof.

The present invention further includes, in one embodiment, a chimeric molecule where at least one of the component molecules is an anti-microbial peptide, protein, analog or active fragment thereof. Such anti-microbial peptides are known and include, for example, defensins, lysozyme, lactoferrin, ITF, magainins, and other natural anti-infectives.

In another embodiment, the present invention includes a chimeric molecule where the first component molecule is a peptide, protein or an active fragment thereof as described herein and the second component molecule is a chemical compound. The chemical compound suitable for use herein is preferably one that has been approved by the FDA, as can be found, for example, at www.FDA.gov, under drugs approved by CDER. In one aspect of this invention, the compound is a hormone such as, for example, testosterone, estrogen, progesterone or analogs or derivatives thereof. In another aspect of the invention, the compound is a toxic compound such as taxol, doxorubicin, cisplatin or analogs or derivatives thereof. In a variation of the invention, the compound is an inhibitor such as a matrix metalloproteases inhibitor, or a chemical anti-infective.

Examples of chimeric molecules that contain two component molecules include, but are not limited to the following combinations: lactoferrin/lactoferrin; lactoferrin/lysozyme; lysozyme/lysozyme; lactoferrin/ITF; lysozyme/ITF; lactoferricin/lactoferricin; ITF/ITF; EGF/EFG; EGF/KGF; KGF/KGF; KGF/PDGF; PDGF/PDGF; α₁-antitrypsin/MMP inhibitor; estrogen/progesterone; antibody/antibody; and analogs, variants and derivatives thereof.

The component molecules of the chimeric molecule may possess different activities. For example, if the component molecule is a matrix metalloproteinase (“MMP”), the component molecule may be selected to alter cell growth, regulate apoptosis, affect cell migration, affect cell-to-cell communication, and affect tumor progression. As an illustration, “MMP-7-generated soluble Fas ligand is effective in killing Fas-expressing tumor cells” disclosed in McCawley, L. J. and Matrisian, L. M in “Matrix metalloproteinase: they're not just for matrix anymore!” Current Opinion in Cell Biology 13:534-540 at 536 (2001). In an alternative embodiment, the chimeric molecule of the present invention may contain a component molecule that is an inhibitor of MMP activity.

The chimeric molecule composition of the present invention is administered to a treated host to achieve a biological effect in the treated host. This biological effect can be diagnostic, prophylactic, therapeutic, anti-infective or nutritional.

The chimeric molecule compositions of the subject invention also find use as therapeutic agents in situations where one wishes to modulate an activity of a subject polypeptide in a host, particularly the activity of the subject polypeptides, or to provide the activity at a particular anatomical site.

The component molecules that can be combined advantageously to form chimeric molecules for delivery or cleavage to different sites in a treated host. In one embodiment, chimeric molecules such as those containing anti-infectives are delivered to the gut of treated hosts. Examples of such component include, but are not limited to: in the GI tract include, for example, but are not limited to: anti-infectives, such as intestinal trefoil factor (“ITF”), and magainins, lactoferrin, lactoferricin, surfactant proteins such as SP-A, SP-D, and lysozyme; anti-inflammatory molecules, such as cyclooxygenase (COX)-2 inhibitor (J. Pharmacol. Exp. Ther. 290: 551 (1999)); anti-cancer molecules, such as DMBT 1 (Deleted in malignant brain tumors 1) which is a secreted tumor suppressor that is deleted in esophageal and digestive tract cancers, as described in Cancer Res. 61: 8880 (2001); nutrition-enhancing molecules, such as milk proteins; and growth factors, such as epidermal growth factor (“EGF”), insulin-like growth factor (“IGF-I”) and keratinocyte growth factor (“KGF”). Component molecules for release in the GI tract include those that are active in the GI tract as well as those that can be transported across the epithelial cells lining the gut, such as IgA. In such an embodiment of the invention, the enzyme is normally present in the gut of the treated host and no other enzymes need be added or administered.

Component molecules can also be advantageously combined for administration to the lungs, for example, using appropriate aerosols to prevent or treat infections, diseases such as cancer, congestive or allergic reactions, or other inflammatory conditions. Anti-infectives suitable for use herein include the surfactant proteins SP-B and SP-C as component molecules.

Further, component molecules may be advantageously administered as chimeric molecules herein to local sites of inflammation such as joints of rheumatoid arthritis patients. These component molecules include, but are not limited to: IL-10, Interleukin 1 receptor antagonist (IL-1Ra), and soluble TNF-α receptor (“solTNFR”) and the like.

In another aspect of the present invention, the chimeric molecules herein are intended for intravenous administration. Examples include but are not limited to: GM-CSF and/or IL-3 for stimulation of multilineage hematopoiesis, and Flt2 and TRAIL for breast carcinoma. In some instances, therapeutic efficacy may be enhanced by designing protease cleavage sites that will be preferentially cleaved at or near the site in the body at which component molecule activity is desired. This is achieved by designing one or more of the cleavage sites in the chimeric molecule to be selectively recognized by a protease the expression and/or the activity of which is locally increased in the condition being treated.

At least one of the component molecules in the chimeric molecule of the present invention, in one embodiment, can be an inhibitory molecule, such as α-Trichosanthin, a eukaryotic ribosome-inactivating protein from Trichosanthes kirilowii, which inhibits the replication of human immunodeficiency virus (“HIV”) (see, for example, Kumagai et al., Proc. Natl. Acad. Sci. 90:427-430 (1993)).

In a preferred embodiment of the present invention, where the chimeric molecule has a first component molecule connected by a linker to a second component molecule, the first component molecule does not inhibit the activity of the second component molecule and vice versa.

In another embodiment, the chimeric molecule of the present invention includes a targeting molecule for directing the chimeric molecule to a location for action in the treated host. The targeting molecule includes a ligand for a receptor, such as a cell surface receptor, for example, or another molecule that has affinity for a location. An example is an anti-CD40 antibody that binds T cells without activating it or a EGF fragment that binds the EGF receptor without activating it. This targeting molecule may take the place of the first component molecule in the present chimeric molecule or may be linked to the first component molecule and be cleavable therefrom.

Optionally, the chimeric molecule of the present invention includes a signal peptide or leader sequence for directing secretion or storage of the chimeric molecule such that when the chimeric molecule is produced in a production host, for example, the chimeric molecule can be secreted from the production host or directed to storage in the production host. Examples of the leader sequence include, but are not limited to: alpha-factor secretory leader from S. cerevisiae, as described in U.S. Pat. No. 4,870,008; or if the chimeric molecule is produced in seeds of monocot plants, signal peptide from a seed storage protein such as from the rice Gt1 and G1b genes, as described in WO 01/83792.

In one embodiment, the chimeric molecule of present invention further includes as a first component molecule, for example, a production host protein or a portion thereof that is highly expressed in the production host (hereafter, “Production Host Peptide”). In one aspect of this invention, the chimeric molecule having a highly expressed Production Host Peptide is linked to one or more, collectively, second component molecules that are typically difficult to express in the absence of the Production Host Peptide. Examples of such second component molecules include small molecule peptides such as ITF, magainins, and other natural anti-infectives. Thus, in one embodiment of the present invention, the chimeric molecule can be represented by the formula: Production Host peptide-(linker-small molecule peptide)ₙ, or Production Host peptide-linker-(small molecule peptide)ₙ where n is a positive integer, of 1 to about 10; optionally, of about 2 to about 7; further optionally, of about 3 to about 5. The small molecule is any small molecule the expression of which is desired and may contain, for example, a sequence of amino acid residues ranging from about 2 to about 60; optionally, from about 5 to about 40; further optionally, from about 7 to about 25; and still optionally, from about 10 to about 20. Optionally, the chimeric molecule of the present invention may also include a moiety that facilitates purification after production from the production host, such as, for example, a histidine tag.

In one embodiment, the present invention further includes a chimeric molecule in which the linker contains a spacer. The spacer may contain just a few amino acid residues, for example, that does not affect enzymatic cleavage of the chimeric molecule at the cleavage site, but yet allows the cleavage site to be exposed for easier cleavage. In another embodiment, the linker contains two cleavage sites, one at each end of the spacer such that the spacer is not attached to any one component molecule.

The chimeric molecule of the present invention can be formulated in any number of ways for delivery into the treated host. In one embodiment, the chimeric molecule is a component of an edible product, such as, for example, when a nucleic acid molecule encoding the chimeric molecule is introduced into a production host. Such edible product includes, but is not limited to: milk (when the production host is an animal such as a goat or a cow), a plant (when the production host is a vegetable such as a tomato), a seed (when the production host is a cereal grain such as rice, wheat, barley, oats, or millet), a microbial cell (when the production host is Lactobacillus or yeast), and derivatives and extracts thereof.

The present invention, therefore, includes methods of delivering chimeric molecules to treated hosts by administering the chimeric molecules orally, buccally, vaginally, rectally, intra-cranially, intra-ventricularly, parenterally or by inhalation. The parenteral route of delivery includes intravenous, intra-arterial, intranasal, intra-muscular, subcutaneous, intra-peritoneal, transdermal or percutaneous.

In another embodiment of the present invention, the chimeric molecule contains polypeptide or nucleic acid vaccines as component molecules, and/or adjuvant molecules as component molecules, or a combination of such. Vaccines can be components of infectious organisms, toxoids, or cancer antigens that are over-expressed by cancer cells.

In one embodiment, the chimeric molecule is not a nucleic acid molecule. However, where the chimeric molecule is a polyprotein, the present invention includes a nucleic acid molecule that encodes the chimeric molecule, as well as a vector that contains the nucleic acid molecule, and a host cell that contains the nucleic acid molecule. Thus, generally, where the chimeric molecule is a polyprotein, the present invention provides a nucleic acid molecule that encodes in the 5′ to 3′ direction: a first component molecule which is linked to a nucleic acid encoding the linker which, in turn, is linked to a nucleic acid molecule encoding a second component molecule. The chimeric molecule herein may be constructed in the form of an expression cassette that contains a promoter and, optionally, a transcription terminator and further optionally, a translation terminator, all inserted into an expression vector that can be used to transfect a suitable host, such as a production host for expression of the chimeric molecule.

In another embodiment of the invention, the chimeric molecule contains component molecules that are nucleic acid molecules, where the linker remains one that contains an enzyme cleavage site as described previously. Such a chimeric molecule may be delivered into a treated host where, upon cleavage the nucleic acid component molecules are transcribed and/or translated to achieve an effect in the treated host, for example.

The present invention further includes compositions containing the chimeric molecule and a pharmaceutically acceptable carrier or excipient. The pharmaceutically acceptable carrier or excipient suitable for use herein is conventional in the art. The composition is formulated in such a way that it is appropriate for the route of administration of the chimeric molecule to the treated host. Hence, the composition may be appropriately formulated for oral delivery, buccal delivery, rectal delivery, vaginal delivery, intracranial delivery, intraventricular delivery, parenteral delivery including intranasal, intravenous, intra-arterial, intraperitoneal, subcutaneous, percutaneous, transdernal delivery and for inhalation.

In another embodiment, the present invention includes a kit that contains the present chimeric molecule composition and instructions for administration of the composition to a treated host. The instructions will typically describe the route of administration of the composition, such as for oral delivery, buccal delivery, rectal delivery, vaginal delivery, intracranial delivery, intraventricular delivery, parenteral delivery including intranasal, intravenous, intra-arterial, intraperitoneal, subcutaneous, percutaneous, transdermal delivery and for inhalation.

Delivery of chimeric molecules having component molecules to a treated host is an efficient method of delivering active molecules to a treated host to achieve a biological effect, when the chimeric molecules can be cleaved in vivo by one or more enzymes present in the treated host. The chimeric molecules of the present invention may be engineered for delivery of active molecules to the host over a longer period of time than individual active component molecules. The chimeric molecule of the present invention may also be engineered for delivery of active molecules to a certain site in the treated host for cleavage and action. The chimeric molecule of the present invention may further be engineered to combine one or more active molecules that can act synergistically or otherwise to address a disease or condition.

The chimeric molecule of the present invention can be made by any process conventional in the art. For example, in one such method, a nucleic acid sequence encoding the first component molecule is linked in the 5′ to 3′ direction to a linker containing at least one cleavage site, which, in turn, is linked to a second component molecule. Typically, linkages are made at appropriate restriction enzyme recognition and cleavage sites, where the different nucleic acid fragments are ligated together in conjunction with regulatory sequences such as promoters, transcription and translation terminators and optionally, enhancers, to create an expression cassette, with or without a selectable marker. Optionally, the selectable marker may be present in a vector into which the expression cassette can be inserted to form an expression vector for transfection into cells (“production hosts”) for production of the chimeric molecules. Examples of how the present chimeric molecule can be made are set forth below for illustrative purposes. They are not intended to be limiting. Conventional techniques are employed though the chimeric molecules, compositions and kits containing such are novel.

Polypeptide Compositions

The term “subject proteins and polypeptides” refers to one embodiment in which the component molecules of the chimeric molecule of the present invention are proteins and polypeptides. These subject proteins and polypeptides can be obtained from naturally occurring sources or produced synthetically, such as by recombinant technology. The sources of naturally occurring proteins and polypeptides will generally depend on the species from which the protein is to be derived. The subject proteins can also be derived from synthetic means, for example, by expressing a recombinant gene encoding a protein of interest in a suitable host. Any convenient protein purification procedures can be employed. For example, a lysate can be prepared from the original source and purified using HPLC, exclusion chromatography, gel electrophoresis, or affinity chromatography. The individual component molecules can be linked together chemically or, expressed as a single polypeptide, for example, by expressing the encoding nucleic acid molecule in a production host.

The invention also provides for use of polypeptide fragments as component molecules in the chimeric molecule herein. In some embodiments, fragments exhibit one or more activities associated with a corresponding naturally occurring polypeptide. Fragments find utility, for example, in generating antibodies to the full-length polypeptide; and in methods of detecting agents that bind to and/or modulate polypeptide activity. Fragments of polypeptides of interest will typically be at least about 10 to 300 amino acids (aa) in length; optionally, the fragment is at least about 25 aa in length; further optionally, the fragment is at least about 50 aa in length, still optionally, the fragment is at least about 75 aa in length; yet still optionally, the fragment is at least about 100 aa in length; still further optionally, the fragment is at least about 200 aa in length. Specific fragments of interest include those with enzymatic activity, those with biological activity, and fragments that bind to other proteins or to nucleic acids.

In addition to naturally occurring proteins, the component molecules of the present chimeric protein can contain polypeptides that vary from naturally occurring forms, such as, variants, including fusion proteins, for example, and analogs and derivatives thereof, where such variants are homologous or substantially similar to the naturally occurring protein. As an example, the fusion proteins can comprise a subject polypeptide, or fragment thereof, and a polypeptide other than a subject polypeptide (“the fusion partner”) fused in-frame at the N-terminus and/or C-terminus of the subject polypeptide, or internally to the subject polypeptide. Such fusion partners may or may not be linked to a component molecule by the present linker.

Suitable fusion partners include, but are not limited to, polypeptides that can bind antibody specific to the fusion partner (for example, epitope tags, such as hemagglutinin, FLAG, and c-myc); polypeptides that provide a detectable signal (for example, a fluorescent protein, for example, a green fluorescent protein, a fluorescent protein from an Anthozoan species; β-galactosidase; luciferase; cre recombinase); polypeptides that provide a catalytic function or induce a cellular response; polypeptides that provide for secretion of the fusion protein from a eukaryotic cell; polypeptides that provide for secretion of the fusion protein from a prokaryotic cell; polypeptides that provide for binding to metal ions (for example, Hisₙ, where n=3-10, such as 6His).

For example, where the fusion partner provides an immunologically recognizable epitope, an epitope-specific antibody can be used to quantitatively detect the level of polypeptide. In some embodiments, the fusion partner provides a detectable signal, and in these embodiments, the detection method is chosen based on the type of signal generated by the fusion partner. For example, where the fusion partner is a fluorescent protein, fluorescence is measured.

Where the fusion partner is an enzyme that yields a detectable product, the product can be detected using appropriate means. For example, the enzyme β-galactosidase, depending on the substrate, can yield a colored product that can be detected with a spectrophotometer, and the fluorescent enzyme, luciferase, can yield a luminescent product detectable with a luminometer.

The polypeptides of the chimeric molecules of the present invention are present in a non-naturally occurring environment, that is, they are separated from their naturally occurring environment. In certain embodiments, the chimeric molecules are substantially purified, such as where the chimeric molecule is present in a composition that is substantially free of other proteins.

The polypeptides of the chimeric molecules of the present invention may be present as an isolate that is substantially free of other proteins or other naturally occurring biological molecules, such as oligosaccharides, polynucleotides, and fragments thereof, and the like. In certain embodiments, the chimeric molecules are at least about 95%, usually at least about 97%, and more usually at least about 97%, optionally, at least about 98% or 99% pure.

Any convenient purification procedures may be employed for the purposes herein. Where suitable, protein purification methodologies are described, for example, in Guide to Protein Purification (Deuthser ed.) (Academic Press, 1990).

Peptides

In some embodiments of the present invention, the component molecule is a peptide. In some embodiments, a peptide exhibits one or more of the following activities: inhibits binding of a subject polypeptide to an interacting protein; inhibits subject polypeptide binding to a second polypeptide molecule; inhibits a signal transduction activity of a subject polypeptide; inhibits an enzymatic activity of a subject polypeptide; inhibits a DNA binding activity of a subject polypeptide. In some embodiments, a peptide has a sequence of from about 3 amino acids to about 50, from about 5 to about 30, or from about 10 to about 25 amino acids of corresponding naturally-occurring protein.

Peptides can include naturally-occurring and non-naturally occurring amino acids. Peptides can comprise D-amino acids, a combination of D- and L-amino acids, and various “designer” amino acids (e.g., β-methyl amino acids, Cα-methyl amino acids, and Nα-methyl amino acids, etc.) to convey special properties. Additionally, peptides can be cyclic. Peptides can include non-classical amino acids in order to introduce particular conformational motifs. Any known non-classical amino acid can be used. Non-classical amino acids include, but are not limited to, 1,2,3,4-tetrahydroisoquinoline-3-carboxylate; (2S,3S)-methylphenylalanine, (2S,3R)-methyl-phenylalanine, (2R,3S)-methyl-phenylalanine and (2R,3R)-methyl-phenylalanine; 2-aminotetrahydronaphthalene-2-carboxylic acid; hydroxy-1,2,3,4-tetrahydroisoquinoline-3-carboxylate; β-carbo line (D and L); HIC (histidine isoquinoline carboxylic acid); and HIC (histidine cyclic urea). Amino acid analogs and peptidomimetics can be incorporated into a peptide to induce or favor specific secondary structures, including, but not limited to, LL-Acp (LL-3-amino-2-propenidone-6-carboxylic acid), a β-turn inducing dipeptide analog; β-sheet inducing analogs; β-turn inducing analogs; α-helix inducing analogs; γ-turn inducing analogs; Gly-Ala turn analogs; amide bond isostere; or tretrazol.

A peptide can be a depsipeptide, which can be linear or cyclic (Kuisle et al., 1999). Peptides can be cyclic or bicyclic. For example, the C-terminal carboxyl group or a C-terminal ester can be induced to cyclize by internal displacement of the —OH or the ester (—OR) of the carboxyl group or ester respectively with the N-terminal amino group to form a cyclic peptide. For example, after synthesis and cleavage to give the peptide acid, the free acid is converted to an activated ester by an appropriate carboxyl group activator such as dicyclohexylcarbodiimide (DCC) in solution, for example, in methylene chloride (CH₂Cl₂), dimethyl formamide (DMF) mixtures. The cyclic peptide is then formed by internal displacement of the activated ester with the N-terminal amine. Internal cyclization as opposed to polymerization can be enhanced by use of very dilute solutions. Methods for making cyclic peptides are well known in the art.

Antibodies

The invention provides chimeric molecules that contain antibodies or active fragments thereof that specifically recognize a particular polypeptide (hereafter, a “target polypeptide”) as one or more component molecules. Suitable antibodies can be produced in a variety of ways conventional in the art, as polyclonal antibodies, monoclonal antibodies, single chain antibodies, and antibody fragments. The antibodies herein include human antibodies, non-human animal antibodies, such as non-human primate antibodies, mouse antibodies, rat antibodies, sheep antibodies, goat antibodies, rabbit antibodies, pig antibodies, cow antibodies, etc., whether in their native form or “humanized,” as conventional in the art. The antibodies herein also include primatized and chimeric antibodies.

The antibodies of the invention can perform diverse functions. They can function as targeting antibodies, neutralizing antibodies, stabilizing antibodies, or enhancing antibodies. They can function as agonists or antagonists of other antibodies. They can mediate ADCC. They can be blocking antibodies, functioning to specifically inhibit the binding of a cognate polypeptide to its ligand or its substrate. Further, antibodies of the invention can specifically inhibit the binding of their cognate peptides as substrates of other molecules.

In one embodiment, polyclonal antibodies are obtained by immunizing a host animal with polypeptides comprising all or a portion of the target polypeptide. For example, to make antibodies against a human target polypeptide, suitable host animals include, but is not limited to: mouse, rat, sheep, goat, hamster, guinea pig, chicken, and rabbit. The origin of the protein immunogen can be any species, including mouse, human, non-human primate, rat, monkey, avian, insect, reptile, or crustacean. The host animal will generally be a different species than the immunogen. Methods of antibody production are well known in the art, as described in Howard and Bethell (2000). Generally, the antibody to be used as a component molecule is compatible with the treated host. For example, if the antibody is to be administered as a component of a chimeric molecule to humans for therapeutic, prophylactic or diagnostic purposes, the antibody is preferably a human antibody or a humanized antibody or active fragments thereof.

The immunogen can comprise the complete protein, or fragments and derivatives thereof. The immunogens, if a protein or parts thereof, can contain post-translation modifications, such as glycosylation, as found on the native target protein. Immunogens comprising extracellular domains of target proteins, such as cancer antigens for example, are produced in a variety of ways known in the art, for example, by expression of cloned genes using conventional recombinant methods, or isolation from tumor cell culture supernatants.

Polyclonal antibodies of the present invention are prepared by conventional techniques. These include immunizing the host animal with the target protein in substantially pure form, comprising less than about 1% contaminant. Alternatively, the host animal can be immunized with whole cells that have been transfected with a nucleic acid molecule encoding the target protein or antigenic portions thereof, such that the whole cells are expressing the immunogen or antigen at a high density, such as membrane proteins, on the cell surface. An example of such is the use of insect cells transfected with baculovirus containing the nucleic acid encoding the target polypeptide or mouse cells transfected with a vector containing such nucleic acid molecules.

To increase the immune response of the host animal to the immunogen, the target protein can be combined with an adjuvant. Suitable adjuvants include, but is not limited to: alum, dextran, sulfate, large polymeric anions, and oil and water emulsions, for example, Freund's adjuvant, complete or incomplete. The target protein can also be conjugated to synthetic carrier proteins or synthetic antigens. The target protein is administered to the host, usually intradermally, subcutaneously, intramuscularly or intraperitoneally, with an initial dosage followed by one or more, usually at least two, additional booster dosages. Following immunization, serum from the immunized host will be collected and tested for antibody production. The immunoglobulin present in the resultant antiserum can be further fractionated using known methods, such as ammonium salt fractionation, or DEAE chromatography.

Monoclonal antibodies of the present invention can also be produced by conventional techniques. Generally, the spleen and/or lymph nodes of an immunized host animal described as above provide a source of plasma cells, which are then immortalized by fusion with myeloma cells to produce antibody-secreting hybridoma cells. The hybridoma cells are cultured and culture supernatants from individual hybridomas are screened using standard techniques to identify clones producing antibodies with the desired specificity. The antibody can be purified from the hybridoma cell supernatants or from ascites fluid present in the host by conventional techniques, e.g. affinity chromatography using antigen bound to an insoluble support, i.e. protein A sepharose. Antibodies produced in such a manner may be subsequently modified or optimized to contain the desired characteristics.

The antibody can be produced as a single chain, instead of the normal multimeric structure of the immunoglobulin molecule. Single chain antibodies have been previously described in. Jost et al. (1994). DNA sequences encoding the variable region of the heavy chain and the variable region of the light chain are ligated to a spacer encoding at least about four small neutral amino acids, i.e. glycine or serine. The protein encoded by this fusion allows the assembly of a functional variable region that retains the specificity and affinity of the original antibody.

The invention also provides “artificial” antibodies, for example, single chain antibodies and antibody fragments produced and selected in vitro. In some embodiments, these antibodies are displayed on the surface of a bacteriophage or other viral particle. In other embodiments, artificial antibodies are present as fusion proteins with a viral or bacteriophage structural protein, including, but not limited to, Ml 3 gene III protein. Methods of producing such artificial antibodies are well known in the art (U.S. Pat. Nos. 5,516,637; 5,223,409; 5,658,727; 5,667,988; 5,498,538; 5,403,484; 5,571,698; and 5,625,033). In some embodiments, the antibody for use herein includes one or more heavy chains, one or more light chains, one or more heavy chains together with one or more light chains, or just the variable regions thereof.

For in vivo use, particularly for injection into humans, in some embodiments it is desirable to decrease the antigenicity of a non-human antibody. An immune response of a treated host against a chimeric molecule containing a non-human antibody may potentially decrease the period of time that the therapy is effective. Methods of humanizing antibodies are known in the art. The humanized antibody can be the product of an animal having transgenic human immunoglobulin constant region genes as described in, for example, International Patent Applications WO 90/10077 and WO 90/04036. Alternatively, the antibody of interest can be engineered by recombinant DNA techniques to substitute the CH1, CH2, CH3, hinge domains, and/or the framework domain with the corresponding human sequence, as described in, for example, WO 92/02190.

Further, genes encoding immunoglobulin can be used in the present invention to make a component of the chimeric molecule or to make a nucleic acid molecule that encodes the chimeric molecule of the present invention. Immunoglobulin genes constructed with immunoglobulin cDNA are known in the art, as described in, for example, Liu et al. (1987a) and Liu et al. (1987b). Messenger RNA is isolated from a hybridoma or spleen or other cell producing such antibody and is used to produce a cDNA library. The cDNA of interest can be amplified by the polymerase chain reaction (“PCR”) using specific primers as described in, for example, U.S. Pat. Nos. 4,683,195 and 4,683,202. Alternatively, a library is made and screened to isolate the sequence of interest. The DNA sequence encoding the variable region of the antibody can be fused to human constant region sequences. The sequences of human constant region (“C region”) genes are known in the art, as described in, for example, Kabat et al., 1991. Human C region genes are readily available from known clones. The choice of isotype will be guided by the desired effector functions, such as complement fixation, or antibody-dependent cellular cytotoxicity. IgG1, IgG3 and IgG4 isotypes, and either of the kappa or lambda light chain constant regions can be used. The chimeric, humanized antibody is then expressed by conventional methods.

In yet other embodiments, the antibodies for use as component molecules in the present chimeric molecule can be fully human antibodies. For example, xenogeneic antibodies, which are identical to human antibodies, can be employed. By xenogenic human antibodies is meant antibodies that are fully human antibodies, with the exception that they are produced in a non-human host that has been genetically engineered to express human antibodies, as described in, for example, WO 98/50433; WO 98,24893 and WO 99/53049.

Antibody fragments suitable for use herein, such as Fv, F(ab′)₂ and Fab, can be prepared by cleavage of the intact protein, e.g. by protease or chemical cleavage. Alternatively, a truncated gene can be designed, for example, a chimeric gene encoding a portion of the F(ab′)₂ fragment that includes DNA sequences encoding the CH1 domain and hinge region of the heavy (“H”) chain, followed by a translational stop codon.

Consensus sequences of H and light (“L”) chains J regions can be used to design oligonucleotides for use as primers to introduce useful restriction sites into the J region for subsequent linkage of variable (“V”) region segments to human C region segments. C region cDNA can be modified by site directed mutagenesis to place a restriction site at the analogous position in the human sequence.

A convenient expression vector for producing antibodies is one that encodes a functionally complete human C_{H} or C_{L} immunoglobulin sequence, with appropriate restriction sites engineered so that any V_{H} or V_{L} sequence can be easily inserted and expressed, such as plasmids, retroviruses, YACs, or EBV derived episomes., In such vectors, splicing usually occurs between the splice donor site in the inserted J region and the splice acceptor site preceding the human C region, and also at the splice regions that occur within the human C_{H} exons. Polyadenylation and transcription termination occur at native chromosomal sites downstream of the coding regions. The resulting chimeric antibody can be joined to any strong promoter, including retroviral LTRs, for example, SV-40 early promoter, as described in, for example, Okayama, et al. (1983); Rous sarcoma virus LTR, as described in, for example, Gorman et al. (1982), and Moloney murine leukemia virus LTR, as described in, for example, Grosschedl et al (1985), or native immunoglobulin promoters.

Nucleic Acid Compositions

The present invention also provides nucleic acid molecules each having an open reading frame that encodes the subject polypeptide or fragments thereof that are capable, under appropriate conditions, of being expressed to produce the subject polypeptides of the chimeric molecule described above. The nucleic acid molecules can be present in the form of a nucleic acid composition that includes a carrier. The term encompasses genomic DNA, cDNA, mRNA, splice variants, antisense RNA, ribozymes, RNAi, peptide nucleic acids, and vectors comprising the subject nucleic acid sequences. Also encompassed in this term are nucleic acids that are homologous or substantially similar or identical to the nucleic acids encoding the subject proteins. Thus, the subject invention provides genes encoding a subject protein, and homologs or derivatives thereof.

The term gene or genomic sequence as used herein is intended to mean either a cDNA or genomic DNA or mRNA that contains an open reading frame encoding specific proteins and polypeptides of the subject invention, with or without introns, with or without adjacent 5′ and 3′ non-coding nucleotide sequences involved in the regulation of expression, up to about 20 kb or beyond the coding region, but possibly further in either direction. The gene may be introduced into an appropriate vector for extrachromosomal maintenance or for integration into a host genome.

The polynucleotides of the present invention may, in one embodiment, include specific transcriptional and translational regulatory sequences, such as promoters, enhancers, etc., including about 1 kb, but possibly more, of flanking genomic DNA at either the 5′ or 3′ end of the transcribed region. In certain embodiments, the genomic DNA may be isolated as a fragment of 100 kbp or smaller; and substantially free of flanking chromosomal sequence. The genomic DNA flanking the coding region, either 3′ or 5′, or internal regulatory sequences as sometimes found in introns, contains sequences required for proper tissue and stage specific expression.

The nucleic acid compositions of the subject invention may encode all or a part of the subject proteins. Double or single stranded fragments may be obtained from the DNA sequence by chemically synthesizing oligonucleotides in accordance with conventional methods, by restriction enzyme digestion, by PCR amplification, etc. For the most part, DNA fragments will be of at least 15 nt, usually at least 18 nt or 25 nt, and may be at least about 50 nt.

When the present chimeric molecule is used as a probe, a subject nucleic acid may include nucleotide analogs that incorporate labels that are directly detectable, such as radiolabels or fluorophores, or nucleotide analogs that incorporate labels that can be visualized in a subsequent reaction, such as various haptens. Common radiolabeled analogs include those labeled with ³²P or ³⁵S, such as α-³²P-dATP, -dTTP, -dCTP, and dGTP; and γ-³⁵S-GTP, α-³⁵S-dATP, and the like. Commercially available fluorescent nucleotide analogs readily incorporated into a subject nucleic acid include deoxyribonucleotides and/or ribonucleotide analogs labeled with Cy3, Cy5, Texas Red, Alexa Fluor dyes, rhodamine, cascade blue, BODIPY, and the like. Haptens that are commonly conjugated to nucleotides for subsequent labeling include biotin, digoxigenin, and dinitrophenyl.

The nucleic acids of the invention can be used for antisense inhibition of transcription or translation, as described below. See, e.g., Phillips (ed.) Antisense Technology, Part B Methods in Enzymology Vol. 314, Academic Press, Inc. (1999); Phillips (ed.) Antisense Technology, Part A Methods in Enzymology Vol. 313, Academic Press, Inc. (1999); Hartmann et al. (eds.) Manual of Antisense Methodology (Perspectives in Antisense Science) Kluwer Law International (1999); Stein et al. (eds.) Applied Antisense Oligonucleotide Technology Wiley-Liss (1998); Agrawal et al. (eds) Antisense Research and Applications Springer-Verlag New York, Inc. (1998).

The subject nucleic acid molecules may also be provided as part of a vector (for example, a polynucleotide construct), a wide variety of which are known in the art and need not be elaborated upon herein. Vectors include, but are not limited to, plasmids; cosmids; viral vectors; human, yeast, bacterial, and P1-derived artificial chromosomes (HAC's, YAC's, BAC's, PAC's, etc.); mini-chromosomes; and the like. Vectors are amply described in numerous publications well known to those in the art, including, e.g., Short Protocols in Molecular Biology, (1999) F. Ausubel, et al., eds., Wiley & Sons; Jones et al. (eds.) Vectors: Cloning Applications: Essential Techniques John Wiley & Son Ltd (1998); Jones et al. (eds.) Vectors: Expression Systems: Essential Techniques John Wiley & Son Ltd (1998). Vectors may provide for expression of the subject nucleic acids; may provide for propagating the subject nucleic acids, or both.

Where a subject nucleic acid is part of a vector or plasmid, the vector or plasmid may be referred to as a “recombinant vector” or a “construct.” Subject constructs are useful for propagating a subject nucleic acid in a production host cell (“cloning vectors”); for shuttling a subject nucleic acid between host cells derived from disparate organisms (“shuttle vectors”); for inserting a subject nucleic acid into a production host cell's chromosome (“insertion vectors”); for expressing sense or antisense RNA transcripts of the invention (for example, in a cell-free system or within a cultured host cell) (“expression vectors”); and for producing a subject polypeptide encoded by a subject nucleic acid in a production host (“expression vectors”).

Vectors typically include at least one origin of replication, at least one site for insertion of heterologous nucleic acid (for example, in the form of a polylinker with multiple, tightly clustered, single cutting restriction endonuclease recognition sites), and at least one selectable marker, although some integrative vectors will lack an origin that is functional in the host to be chromosomally modified, and some vectors will lack selectable markers.

For a nucleic acid molecule that encodes the chimeric molecule of the present invention or for a chimeric molecule containing nucleic acid molecules as component molecules, the nucleic acid molecules typically contain genes or polynucleotides that are isolated and obtained in substantial purity. Usually, the DNA will be obtained substantially free of other nucleic acid sequences that do not include a sequence or fragment thereof of the subject genes, generally being at least about 50%, usually at least about 90% pure and are typically “recombinant”, that is, flanked by one or more nucleotides with which it is not normally associated on a naturally occurring chromosome.

Antisense Oligonucleotides

In yet another embodiment of the invention, a component molecule for intracellular administration of the chimeric molecule to a treated host is an agent that modulates, and generally decreases or down regulates, the expression of the gene encoding the target protein in the host, such as, for example antisense molecules, ribozymes, or RNAi.

Anti-sense reagents include antisense oligonucleotides (ODN), that is, synthetic ODN having chemical modifications from native nucleic acids, or nucleic acid constructs that express such anti-sense molecules as RNA. The antisense sequence is complementary to the mRNA of the targeted gene, and inhibits expression of the targeted gene products. Antisense molecules inhibit gene expression through various mechanisms, e.g. by reducing the amount of mRNA available for translation, through activation of RNAse H, or steric hindrance. One or a combination of antisense molecules can be administered, where a combination can comprise multiple different sequences.

Antisense molecules can be produced by expression of all or a part of the target gene sequence in an appropriate vector, where the transcriptional initiation is oriented such that an antisense strand is produced as an RNA molecule. Alternatively, the antisense molecule is a synthetic oligonucleotide. Antisense oligonucleotides will generally be at least about 7, usually at least about 12, more usually at least about 20 nucleotides in length, and not more than about 500, and not more than about 50, and not more than about 35 nucleotides in length, where the length is governed by efficiency of inhibition, specificity, including absence of cross-reactivity. Short oligonucleotides, of from 7 to 8 bases in length, can be strong and selective inhibitors of gene expression as described in, for example, Wagner et al., (1996).

A specific region or regions of the endogenous sense strand mRNA sequence is chosen to be complemented by the antisense sequence. Selection of a specific sequence for the oligonucleotide can use an empirical method, where several candidate sequences are assayed for inhibition of expression of the target gene in an in vitro or animal model. A combination of sequences can also be used, where several regions of the mRNA sequence are selected for antisense complementation.

Antisense oligonucleotides can be chemically synthesized by methods known in the art, as described in, for example, Wagner et al., (1993); Milligan et al., (1993) Oligonucleotides can be chemically modified from the native phosphodiester structure to increase their intracellular stability and binding affinity.

As an alternative to anti-sense inhibitors, catalytic nucleic acid compounds, e.g. ribozymes, or anti-sense conjugates can be used to inhibit gene expression. Ribozymes can be synthesized in vitro, or can be encoded in an expression vector, from which the ribozyme is synthesized in the targeted cell as described in, for example, WO 9523225 and Beigelman et al., (1995). Examples of oligonucleotides with catalytic activity are described in WO 9506764. Conjugates of anti-sense ODN with a metal complex, e.g. terpyridylCu(II), capable of mediating mRNA hydrolysis are described in Bashkin et al., (1995).

Interfering RNA

In some embodiments, a component molecule is an interfering RNA (RNAi). RNA interference provides a method of silencing eukaryotic genes. Double stranded RNA can induce the homology-dependent degradation of its cognate mRNA in C. elegans, fungi, plants, Drosophila, and mammals (Gaudilliere, et al., 2002). Use of RNAi to reduce a level of a particular mRNA and/or protein is based on the interfering properties of double-stranded RNA derived from the coding regions of a gene. The technique is an efficient high-throughput method for disrupting gene function (O'Neil, 2001).

In one embodiment of the invention, complementary sense and antisense RNAs derived from a substantial portion of the subject polynucleotide are synthesized in vitro. The resulting sense and antisense RNAs are annealed in an injection buffer, and the double-stranded RNA injected or otherwise introduced into the subject, i.e. in food or by immersion in buffer containing the RNA (WO99/32619). In another embodiment, dsRNA derived from a gene of the present invention is generated in vivo by simultaneously expressing both sense and antisense RNA from appropriately positioned promoters operably linked to coding sequences in both sense and antisense orientations.

Preparation of the Subject Polypeptides

In addition to the plurality of uses described in greater detail in following sections, the subject nucleic acid compositions find use in the preparation of the polypeptides of the chimeric molecule of the present invention. Generally, for expression of a polynucleotide, an expression cassette may be employed that comprises an expression vector that contains a transcriptional and translational initiation region, which may be inducible or constitutive, where the coding region is operably linked under the transcriptional control of the transcriptional initiation region, and a transcriptional and translational termination region.

The chimeric molecule of the present invention, hence, can be made by any conventional techniques customary in the art. It is to be understood that, unless otherwise expressly provided, the present invention is not limited to any particular method of making the chimeric molecules herein. For example, the chimeric molecule of the present invention can be made by recombinant techniques such as described in the patents and publications cited herein or as described in Sambrook et al. (1989) Molecular Cloning: A Laboratory Manual (Second Edition), Cold Spring Harbor Press, Plainview, N.Y.; and Ausubel F. M. et al. (1993) Current Protocols in Molecular Biology, John Wiley & Sons, New York, N.Y. Further, biological protocols may be accessed via websites such as: http://www.bioprotocol.com. The chimeric molecules herein can also be made by laboratory synthesis, for example, by creating several polypeptide sequences, polynucleotide sequences or chemical entities and linking such sequences or molecules together in vitro.

In one embodiment of the present invention, a DNA molecule that encodes the present chimeric molecule can be incorporated into an expression cassette that can be expressed in a production host for production of the chimeric molecule. The expression cassette will include transcription and translation regulatory sequences such as a promoter, transcription initiation and termination sequences as well as translation initiation and termination sequences. An enhancer may or may not be present in cis or trans position. As an illustration, in the 5′ to 3′ direction, a DNA fragment containing transcription and translation regulatory sequences can be linked to DNA encoding the chimeric molecule, which contains: a first DNA fragment that encodes the first component molecule which, in turn, can be linked to a second DNA fragment that encodes a linker containing a cleavage site which, in turn, can be linked to a third DNA fragment that encodes a second component molecule, followed by translation and transcription termination sequences. Linkages are typically made at suitable restriction endonuclease restriction sites and the different fragments ligated together, for example, with DNA ligase. The expression cassette can be part of a plasmid or viral vector. Vectors that are commonly used include the Gateway vectors (www.Invitrogen.com) and the Creator vectors (www.bdbioscience.com).

Thus, the instant invention provides methods of producing the subject polypeptides of the present invention, including the chimeric molecule herein when the molecule is a polyprotein, and the component molecules herein when the component molecules are peptides or polypeptides or active fragments thereof. The methods generally involve introducing a nucleic acid construct as above into a host cell either for in vivo or in vitro production. For in vitro production of the chimeric molecules, the host cell is cultured in vitro under conditions that are suitable for expression of the nucleic acid construct and production of the encoded subject polypeptide; and harvesting the subject polypeptide, for example, from the culture medium, or from within the host cell (for example, by disrupting the host cell), or both.

The instant invention also provides methods of producing a subject polypeptide using cell-free in vitro transcription/translation methods, which are well known in the art, for example, by use of a rabbit reticulocyte cell-free lysates, frog oocyte lysates, wheat germ lysates, bacterial lysates, etc., as described in, for example, WO 00/68412, WO 01/27260, WO 02/24939, WO 02/38790, WO 91/02076, and WO 91/02075.

The instant invention further provides methods of producing a subject polypeptide in vivo, for example, in a transgenic animal, as described in, for example WO 93/25567.

The instant invention further provides host cells, for example, recombinant host cells that comprise a subject nucleic acid, and host cells that comprise a subject recombinant vector. Subject host cells can be in in vitro culture, or may be part of a multicellular organism. Host cells are described in more detail below.

Optionally, a signal, leader or transit sequence encoding a signal, leader or transit peptide for directing the chimeric molecule to certain compartments or organelles in the production host for processing and/or secretion may be inserted between the regulatory sequences and the first DNA fragment that encodes the first component molecule, as appropriate. For example, if the production host is a yeast cell, a signal or leader sequence that would direct the fusion protein to the Golgi apparatus for processing and secretion would be appropriate. Such signal sequences can be from the pre- or pro-sequences of secreted proteins, such as the alpha factor of yeast, as described in U.S. Pat. No. 4,870,008.

Alternatively, if the production host is a plant, such as a rice plant, an expression cassette may be constructed for expression of the chimeric molecule in the plant seeds, using regulatory sequences and leader sequences as described in WO 99/16890. Expression cassettes for the production of the present chimeric molecule in a plant production host can also be made as described in WO 00/04146.

An expression cassette for the production of the chimeric molecule of the present invention in a fungal host such as Aspergillus can also be made, for example, as described in WO 97/45156 and WO 93/22348.

The chimeric molecule of the present invention can further be made with all or a portion of a protein that is highly expressed in the production host. Expression cassettes containing such are described, for example in U.S. Pat. No. 4,828,988, U.S. Pat. No. 5,292,646.

Expression vectors suitable for use herein for the production of nucleic acid molecules encoding the chimeric molecules generally have convenient restriction sites located near the promoter sequence to provide for the insertion of the nucleic acid molecules. A selectable marker operative in the expression host may optionally be present in such a construct. Expression vectors may additionally contain nucleic molecules encoding fusion partners, where the fusion partners provides additional functionality, i.e. increased protein synthesis, a leader sequence for secretion, stability, reactivity with defined antisera, an enzyme marker, e.g. β-galactosidase, etc.

Expression cassettes suitable for use herein may be prepared that comprises a transcription initiation region, the gene or fragment thereof, and a transcriptional termination region. Of particular interest is the use of sequences that allow for the expression of functional epitopes or domains, usually at least about 8 amino acids in length, more usually at least about 15 amino acids in length, to about 25 amino acids, or any of the above-described fragment, and up to the complete open reading frame of the gene. After introduction of the DNA into a production host, the cells containing the construct may be selected by means of a selectable marker, the cells expanded and then used for expression.

The chimeric molecules that contain proteins, polypeptides, including antibodies as component molecules, may be expressed in prokaryotes or eukaryotes in accordance with conventional ways, depending upon the purpose for expression. For large scale production of the protein, a unicellular organism, such as E. coli, B. subtilis, S. cerevisiae, Pichia pastoris, or Kluyveromyces lactis, Aspergillus oryza, insect cells in combination with baculovirus vectors such as SF9 cells or High Five cells, or cells of a higher organism such as plants and vertebrates, particularly mammals, for example, COS 7 cells, may be used as the expression host cells. In some situations, it is desirable to express the gene in eukaryotic cells, where the encoded protein will benefit from native folding and post-translational modifications. Suitable plant cells include, but are not limited to, a dicot, such as a tobacco plant, tomato plant, or a monocot, including seeds thereof, such as cereal grains: oats, rice, wheat, barley, sorghum and other edible plants. The combination of promoters, enhancers, terminators, and vectors can be optimized for expression in each host. Small peptides can also be synthesized in the laboratory.

When any of the above host cells described in the Examples, or other appropriate host cells or organisms, are used to replicate and/or express the chimeric molecules containing polynucleotides or nucleic acids of the invention, the resulting replicated nucleic acid, RNA, expressed protein or polypeptide, is within the scope of the invention as a product of the production host cell or organism. The product is recovered by any appropriate means known in the art. For example, a lysate may prepared from the original source, (for example, a cell expressing endogenous subject polypeptide, or a cell comprising the expression vector expressing the subject polypeptide(s)), and purified using HPLC, exclusion chromatography, gel electrophoresis, affinity chromatography, and the like.

Further description of expression systems suitable for use herein include, for example, U.S. Pat. Nos. 4,745,069; 4,828,988; 6,312,923; 6,342,375; 6,235,878; RE 37,343; 6,068,994; 6,080,559; 5,695,9856, 277,633; 6,232,105; 6,222,094; 5,888,814; 5,981,275; 6,025,540; 5,750,172; 6,329,137; U.S. Pat. No. 6,303,369.

Where the chimeric molecule herein is expressed in a plant production host, the chimeric molecule may be targeted for expression in the leaves or shoots of plants. Alternatively, the chimeric molecule can be targeted for expression in the grains or seeds of plants, such as monocots like cereal plants, including rice, wheat, barley, oats, millet, corn and sorghum. The chimeric molecule expressed in seeds of plant production hosts can be processed in such a way that the activity of the chimeric molecule is preserved or substantially maintained. Thus, extracts of the chimeric molecule containing seeds can be made and the extracts can be incorporated in food as food supplements or nutritional additives. Alternatively, the seeds or seed extracts can be processed using a low temperature process, such as by adding the extracts to a malting brew under conditions where the starch in the grains becomes converted to malt syrup and the polypeptides remain substantially intact. If such a system for producing the chimeric molecule herein is used, the cleavage site in the chimeric molecule has to be designed such that it is not activated during the processing of the seeds or extracts.

Compositions

The present invention further provides compositions, including pharmaceutical compositions, comprising the chimeric molecules of the present invention. These compositions may include a buffer, which is selected according to the desired use of the chimeric molecules, and may also include other substances appropriate for the intended use. Those skilled in the art can readily select an appropriate buffer, a wide variety of which are known in the art, suitable for an intended use. In some instances, the composition can comprise a pharmaceutically acceptable excipient, a variety of which are known in the art and need not be discussed in detail herein. Pharmaceutically acceptable excipients suitable for use herein are described in a variety of publications, including, for example, A. Gennaro (1995) “Remington: The Science and Practice of Pharmacy”, 19th edition, Lippincott, Williams, & Wilkins.

The compositions herein are formulated in accordance to the mode of potential administration. Thus, if the composition is intended to be administered intranasally or by inhalation, the composition may be a converted to a powder form, as conventional in the art, for such purposes. Other formulations, such as for oral or parenteral delivery, are also used as conventional in the art.

Excipients and Formulations

In some embodiments, compositions are provided in formulation with pharmaceutically acceptable excipients, a wide variety of which are known in the art, as described in, for example, Gennaro, 2000; Ansel et al., 1999; Kibbe et al., 2000. Pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

In pharmaceutical dosage forms, the chimeric molecules of the invention can be administered in the form of their pharmaceutically acceptable salts, or they can also be used alone or in appropriate association, as well as in combination, with other pharmaceutically active compounds. The following methods and excipients are merely exemplary and are in no way limiting.

For oral preparations, the chimeric molecules can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

Suitable excipient vehicles are, for example, water, saline, dextrose, glycerol, and ethanol, and combinations thereof. In addition, if desired, the vehicle can contain minor amounts of auxiliary substances such as wetting or emulsifying agents or pH buffering agents. Actual methods of preparing such dosage forms are known, or will be apparent, to those skilled in the art (Remington, 1985). The composition or formulation to be administered will, in any event, contain a quantity of the chimeric molecules adequate to achieve the desired state in the subject being treated.

The chimeric molecules can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

The chimeric molecules can be utilized in aerosol formulation to be administered via inhalation. The compounds of the present invention can be formulated into pressurized acceptable propellants such as dichlorodifluoromethane, propane, or nitrogen.

Furthermore, the chimeric molecules can be made into suppositories by mixing with a variety of bases such as emulsifying bases or water-soluble bases. The compounds of the present invention can be administered rectally via a suppository. The suppository can include vehicles such as cocoa butter, carbowaxes and polyethylene glycols, which melt at body temperature, yet are solidified at room temperature.

Unit dosage forms for oral or rectal administration such as syrups, elixirs, and suspensions can be provided wherein each dosage unit, for example, teaspoonful, tablespoonful, tablet or suppository, contains a predetermined amount of the composition containing one or more inhibitors. Similarly, unit dosage forms for injection or intravenous administration can comprise the inhibitor(s) in a composition as a solution in sterile water, normal saline or another pharmaceutically acceptable carrier.

Diagnostic, Prophylactic, Therapeutic and Nutrition-Enhancing Methods

The instant invention provides various diagnostic, prophylactic therapeutic, and nutrition-enhancing methods, where the methods include administering to a treated host an effective amount of the chimeric molecule of the present invention or a composition containing such, where the component molecules have diagnostic, prophylactic, therapeutic and nutrition-enhancing activity.

In some embodiments, the methods include administering a chimeric molecule or a composition containing a chimeric molecule to a treated host, where the component molecules are capable of binding to a biological molecule for diagnostic purposes. For example, the component molecules can be polynucleotides carrying a detectable label that are capable of binding to circulating nucleic acid molecules encoding self antigens or antigens of infectious organisms.

In some embodiments, the methods include administering a chimeric molecule or a composition containing a chimeric molecule to a treated host, where the component molecules are vaccines, for prophylactic purposes. For example, the component molecules can be polypeptide or DNA vaccine. Such a vaccine may be particularly advantageous where the component molecules are small peptides that would otherwise be degraded.

In some embodiments, the methods include administering a chimeric molecule or a composition containing such to a treated host, where the component molecules have therapeutic value, such as modulating, such as activating, increasing or inhibiting, a biological activity of a protein or receptor in the treated host. In some embodiments, the present methods include modulating an enzymatic activity of a protein in the treated host. In other embodiments, methods of modulating a signal transduction activity of a protein in the treated host are provided. In other embodiments, methods of modulating interaction of a subject protein with another, interacting protein or other macromolecule (e.g., DNA, carbohydrate, lipid) are provided.

In some embodiments, the methods herein include administering a chimeric molecule or a composition containing such to a treated host, to enhance the nutrition of the treated host, either in terms of providing component molecules that have nutritional value or providing anti-infectives to optimize the food value.

The present invention also provides for delivering or administering a chimeric molecule or a composition containing such to a treated host, where the chimeric molecule advantageously reduces degradation of the component molecules.

The present invention further provides for delivering or administering a chimeric molecule or a composition containing such to a treated host, where the chimeric molecule advantageously increases availability of the component molecules.

A variety of hosts are treatable according to the subject methods, including human and non-human animals. Generally such hosts are “mammals” or “mammalian,” where these terms are used broadly to describe organisms which are within the class mammalia, including the orders carnivore (e.g., dogs and cats), rodentia (e.g., mice, guinea pigs, and rats), and other mammals, including cattle, goats, sheep, rabbits, and pigs, and primates (e.g., humans, chimpanzees, and monkeys). In many embodiments, the hosts will be humans. Animal models are of interest for experimental investigations, providing a model for treatment of human disease.

Formulations, Dosages, and Routes of Administration

An effective amount of the chimeric molecule (containing small molecule, antibody specific for a subject polypeptide, or a subject polypeptide as component molecule) is administered to the host, where “effective amount” means a dosage sufficient to produce a desired result. For example, in some embodiments, the desired result is at least a reduction in a given biological activity of a subject polypeptide as compared to a control, while in other embodiments, the desired result is an increase in the level of active subject polypeptide (in the individual, or in a localized anatomical site in the individual), as compared to a control. Also as an example, in some embodiments, the desired result is at least a reduction in enzymatic activity of a subject polypeptide as compared to a control, while in other embodiments, the desired result is an increase in the level of enzymatically active subject polypeptide (in the individual, or in a localized anatomical site in the individual), as compared to a control.

Typically, the compositions of the instant invention will contain from less than 1% to about 95% of the active ingredient, preferably about 10% to about 50%. Generally, between about 100 mg and 500 mg will be administered to a child and between about 500 mg and 5 grams will be administered to an adult. Administration is generally by injection and often by injection to a localized area. The frequency of administration will be determined by the care giver based on patient responsiveness. Other effective dosages can be readily determined by one of ordinary skill in the art through routine trials establishing dose response curves.

In order to calculate the amount of chimeric molecules or component molecules, those skilled in the art could use readily available information with respect to the amount of component molecules necessary to have a the desired effect. The amount of an component molecules necessary to increase a level of active subject polypeptide can be calculated from in vitro experimentation. The amount of component molecules will, of course, vary depending upon the particular component molecules used.

In the subject methods, the chimeric molecule may be administered to the host using any convenient means capable of resulting in the desired activity. Thus, the chimeric molecule can be incorporated into a variety of formulations for administration to the host. More particularly, the chimeric molecule of the present invention can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols.

As such, administration of the chimeric molecule can be achieved in various ways, such as oral, buccal, rectal, parenteral, including intranasal, intravenous, intra-arterial, intraperitoneal, intradermal, transdermal, subcutaneous, percutaneous, intracheal, intracardiac, intraventricular, intracranial, etc., and administration by implantation. The agents may be administered daily, weekly as appropriate or as conventionally determined.

The term “unit dosage form,” as used herein, refers to physically discrete units suitable as unitary dosages for human and animal subjects, each unit containing a predetermined quantity of compounds of the present invention calculated in an amount sufficient to produce the desired effect in association with a pharmaceutically acceptable diluent, carrier or vehicle. The specifications for the novel unit dosage forms of the present invention depend on the particular compound employed and the effect to be achieved, and the pharmacodynamics associated with each compound in the host.

Where the chimeric molecule contains as component molecules a polypeptide, polynucleotide, analog or mimetic thereof, e.g. antisense composition, it may be introduced into tissues or host cells by any number of routes, including viral infection, microinjection, or fusion of vesicles. Jet injection may also be used for intramuscular administration, as described by Furth et al. (1992), Anal Biochem 205:365-368. The DNA may be coated onto gold microparticles, and delivered intradermally by a particle bombardment device, or “gene gun” as described in the literature (see, for example, Tang et al. (1992), Nature 356:152-154), where gold microprojectiles are coated with the therapeutic DNA, then bombarded into skin cells.

Those of skill will readily appreciate that dose levels can vary as a function of the specific compound, the severity of the symptoms and the susceptibility of the subject to side effects. Preferred dosages for a given compound are readily determinable by those of skill in the art by a variety of means.

By treatment is meant at least an amelioration of the symptoms associated with the pathological condition afflicting the host, where amelioration is used in a broad sense to refer to at least a reduction in the magnitude of a parameter, e.g. symptom, associated with the pathological condition being treated, such as inflammation and pain associated therewith. As such, treatment also includes situations where the pathological condition, or at least symptoms associated therewith, are completely inhibited, e.g. prevented from happening, or stopped, e.g. terminated, such that the host no longer suffers from the pathological condition, or at least the symptoms that characterize the pathological condition.

Kits with unit doses of the active agent, usually in oral or injectable doses, are provided. In such kits, in addition to the containers containing the unit doses will be an informational package insert describing the use and attendant benefits of the drugs in treating pathological condition of interest. Preferred compounds and unit doses are those described herein above.

In one embodiment, the chimeric molecule containing the antibodies as component molecules are administered for the treatment of cancer, or proliferative disorder, or immune disorder or metabolic disorder, to subjects in need of such treatment. Such antibodies may be administered by injection systemically, such as by intravenous injection; or by injection or application to the relevant site, such as by direct injection into the tumor, or direct application to the site when the site is exposed in surgery; by topical application, such as if the disorder is on the skin, for example. Such antibodies may be administered alone or in combination with other agents, such as cytotoxic agents.

Tumors which may be treated using the methods of the instant invention include carcinomas, e.g. colon, rectum, prostate, breast, melanoma, ductal, endometrial, stomach, pancreatic, mesothelioma, dysplastic oral mucosa, invasive oral cancer, non-small cell lung carcinoma (“NSCL”), transitional and squamous cell urinary carcinoma, etc.; neurological malignancies, e.g. neuroblastoma, glioblastoma, astrocytoma, gliomas, etc.; hematological malignancies, e.g. childhood acute leukaemia, non-Hodgkin's lymphomas, chronic lymphocytic leukaemia, malignant cutaneous T-cells, mycosis fungoides, non-MF cutaneous T-cell lymphoma, lymphomatoid papulosis, T-cell rich cutaneous lymphoid hyperplasia, bullous pemphigoid, discoid lupus erythematosus, lichen planus, etc.; gynecological cancers, e.g., cervical and ovarian; testicular cancers; liver cancers including hepatocellular carcinoma (“HCC”) and tumor of the biliary duct; multiple myelomas; tumors of the esophageal tract; other lung tumors including small cell and clear cell; Hodgkin's lymphomas; sarcomas in different organs; and the like.

In other embodiments, e.g., where the disease or condition to be treated is inflammation or immune function, the invention provides chimeric molecules of the present invention, containing polynucleotides, polypeptides, antibodies, small molecules, etc., for treating such inflammation or immune disorder. Disease states which are treatable using formulations of the invention include various types of arthritis such as rheumatoid arthritis and osteoarthritis, various chronic inflammatory conditions of the skin, such as psoriasis, inflammatory bowel disease (“IBD”), insulin-dependent diabetes, autoimmune diseases such as multiple sclerosis (“MS”) and systemic lupus erythematosis (“SLE”), allergic diseases, transplant rejections, adult respiratory distress syndrome, atherosclerosis, ischemic diseases due to closure of the peripheral vasculature, cardio vasculature, and vasculature in the central nervous system (“CNS”). After reading the present disclosure, those skilled in the art will recognize other disease states and/or symptoms which might be treated and/or mitigated by the administration of formulations of the present invention.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

### Expression in Bacteria

The chimeric molecule herein can be expressed in a bacterial production host. Expression systems in bacteria include those described in Chang et al., Nature (1978) 275:615; Goeddel et al., Nature (1979) 281:544; Goeddel et al., Nucleic Acids Res. (1980) 8:4057; EP 0 036,776; U.S. Pat. No. 4,551,433; DeBoer et al., Proc. Natl. Acad. Sci. (USA) (1983) 80:21-25; and Siebenlist et al., Cell (1980) 20:269.

### Example 2

### Expression in Yeast

The chimeric molecules herein can be expressed in a yeast production host. Expression systems in yeast include those described in Hinnen et al., Proc. Natl. Acad. Sci. (USA) (1978) 75:1929; Ito et al., J. Bacteriol. (1983) 153:163; Kurtz et al., Mol. Cell. Biol. (1986) 6:142; Kunze et al., J. Basic Microbiol. (1985) 25:141; Gleeson et al., J. Gen. Microbiol. (1986) 132:3459; Roggenkamp et al., Mol. Gen. Genet. (1986) 202:302; Das et al., J. Bacteriol. (1984) 158:1165; De Louvencourt et al., J. Bacteriol. (1983) 154:737; Van den Berg et al., Bio/Technology (1990) 8:135; Kunze et al., J. Basic Microbiol. (1985) 25:141; Cregg et al., Mol Cell. Biol. (1985) 5:3376; U.S. Pat. Nos. 4,837,148 and 4,929,555; Beach and Nurse, Nature (1981) 300:706; Davidow et al., Curr. Genet. (1985) 10:380; Gaillardin et al., Curr. Genet. (1985) 10:49; Ballance et al., Biochem. Biophys. Res. Commun. (1983) 112:284-289; Tilbum et al., Gene (1983) 26:205-221; Yelton et al., Proc. Natl. Acad. Sci. (USA) (1984) 81:1470-1474; Kelly and Hynes, EMBO J. (1985) 4:475479; EP 0 244,234; and WO 91/00357.

### Example 3

### Expression in Baculovirus Expression System

The chimeric molecules herein can be expressed in an insect cell production host. Expression of heterologous genes in insects is accomplished as described in U.S. Pat. No. 4,745,051; Friesen et al., “The Regulation of Baculovirus Gene Expression”, in: The Molecular Biology Of Baculoviruses (1986) (W. Doerfler, ed.); EP 0 127,839; EP 0 155,476; and Vlak et al., J. Gen. Virol. (1988) 69:765-776; Miller et al., Ann. Rev. Microbiol. (1988) 42:177; Carbonell et al., Gene (1988) 73:409; Maeda et al., Nature (1985) 315:592-594; Lebacq-Verheyden et al., Mol. Cell. Biol. (1988) 8:3129; Smith et al., Proc. Natl. Acad. Sci. (USA) (1985) 82:8844; Miyajima et al., Gene (1987) 58:273; and Martin et al., DNA (1988) 7:99. Numerous baculoviral strains and variants and corresponding permissive insect host cells from hosts are described in Luckow et al., Bio/Technology (1988) 6:47-55, Miller et al., Generic Engineering (1986) 8:277-279, and Maeda et al., Nature (1985) 315:592-594.

### Example 4

### Expression in Mammalian Cells

The chimeric molecules can also be expressed in mammalian cells. Mammalian expression is accomplished as described in Dijkema et al., EMBO J. (1985) 4:761, Gorman et al., Proc. Natl. Acad. Sci. (USA) (1982) 79:6777, Boshart et al., Cell (1985) 41:521 and U.S. Pat. No. 4,399,216. Other features of mammalian expression are facilitated as described in Ham and Wallace, Meth. Enz. (1979) 58:44, Barnes and Sato, Anal. Biochem. (1980) 102:255, U.S. Pat. Nos. 4,767,704, 4,657,866, 4,927,762, 4,560,655, WO 90/103430, WO 87/00195, and U.S. RE 30,985.

### Example 5

### A Chimeric Molecule Containing EFG and TFF

Component molecules that can be advantageously released in the gut or intestinal tract of a treated host include those having anti-microbial activities, such as lactoferrin and lysozyme, and those with protective or healing properties for mucosal tissues, for example, human epidermal growth factor (EGF) and the trefoil factor family peptides: TFF1 (formerly pS2), TFF2 (formerly hSP), and TFF3 (formerly intestinal trefoil factor). In such an embodiment of the invention, the cleavage site of the chimeric molecule is designed to be proteolyzed by an enzyme that is active in or on the surface of the digestive tract of the treated host. For example, human enterokinase is active in the duodenal and jejunal mucosa.

In a preferred embodiment of the invention the chimeric molecule comprises one or more copies of both human EGF and TFF2, each joined by a linker containing the enterokinase cleavage site. The recombinant product is administered orally as a purified drug in a pharmaceutically acceptable carrier or as a nutraceutical expressed in transgenic cow, sheep, or goat milk or in transgenic plant products, such as rice. The patient population expected to benefit from such treatment includes but is not limited to those with acute gastro-intestinal inflammatory diseases such as ulcerative colitis and flare-ups of Crohn's disease, as well as those with chronic forms of these diseases. Patients with ulcers or mucosal damage resulting from infections or consumption of alcohol or NSAIDs are also predicted to benefit from such treatment.

TFF2 and EGF appear to be well suited to be components of an orally delivered recombinant chimeric molecule for several reasons. They are both normally produced in the digestive tract, are both stable to those conditions, and appear to synergize biologically (Oertel, M., et al., Am J Respir Cell Mol Biol 25: 418, 2001). EGF is a molecule with broad biological potential and has been tested clinically in patients with necrotizing enterocolitis, Zollinger-Ellison Syndrome, gastrointestinal ulceration, and congenital microvillus atrophy (Guglietta., A., et al., Eur J Gastroenterol Hepatol 7:945-50, 1995). EGF is mitogenic toward gastrointestinal mucosa and inhibits gastric acid secretion, both of which are believed to speed healing. Recombinant human EGF has been reported to be orally active in the treatment of duodenal ulcers in a placebo-controlled, double-blind clinical study (Palomino, A., et al., Scand J Gastroenterol 35:1066-22, 2000).

A chimeric molecule consisting of N-terminal EGF, followed by a linker containing the enterokinase cleavage site and TFF2 may improve the production and use of recombinant EGF in several ways. Mature recombinant EGF is about 6 kD in MW and when produced in a number of production hosts has been reported to lose activity due to C-terminal processing during production or purification (Engler, D., et al., J Biol Chem 263:12384-90, 1986). C-terminal fusion components on EGF may retard or eliminate such unwanted processing, while possibly increasing expression levels. Fusion of EGF to TFF2 should also retain EGF activity in the desired locations for longer periods as a consequence of the reported ability of TFFs to bind mucin glycoproteins in the mucosa of the stomach and small intestine (Poulson, S., et al., Gut 43:240-47, 1998). Similarly, the 8-min half-life of intravenously administered recombinant EGF (Calnan, D, et al., Gut 47:622-27,2000) would be predicted to increase in this fusion construct as a consequence of TFF2 mucin binding and an increase in MW.

The TFF2 component of the above chimericmolecule construct is about 12 to 14 kD in MW, depending upon whether or not the production host used is capable of fully glycosylating the molecule at its single N-linked site. In rat models, recombinant TFF2 accelerates gastric ulcer healing via both subcutaneous and oral routes (Poulsen, S., et al., Gut 45:516-22,1999), enhances mucosal blood flow and inhibits gastric secretion (Konturek, P., et al., Regul Pept 68:71-9, 1997), and stimulates migration of human monocytes (Cook. G., et al., FEBS Lett 456:155-59, 1999). As predicted by these observations, TFF2 knock-out mice showed decreased gastric mucosal growth, increased acid secretion, and increased susceptibility to gastric ulceration after treatment with indomethacin (Farrell, J., et al., J Clin Invest 109:193-204, 2002). In diverse ulcerative conditions, glandular structures are formed that produce all three trefoil factors as well as EGF, presumably to facilitate local healing (Poulsom, R., Baillieres Clin Gastroenterol 110:113-34, 1996). In summary, TFF2 can work effectively with EGF in a chimeric molecule with improved properties for recombinant production as well as efficacy against the gastrointestinal conditions listed above.

Saccharomyces cerevisiae has been used successfully to produce recombinant forms of both EGF (Herber Biote SA, Havana, Cuba) and TFF2 (Thim, L., et al., FEBS Lett 318:345-52, 1993). Escherichia coli has been used successfully to produce refolded recombinant EGF (Lee, J., et al., Biotechnol Appl Biochem 31:245-48, 2000) and TFF1, a molecule homologous to TFF2, but having one rather than two cystein-rich trefoil domains. Thus, it should be possible to use one or both of these production hosts to make useful amounts of an active recombinant EGF/linker/TFF2 chimeric molecule.

### Example 6

### Preparation of a Chimeric Molecule Comprising Human Epidermal Growth Factor, a Linker Containing the Enterokinase Cleavage Site, and Human TFF2

In accordance with the invention, an EGF/enterokinase cleavage-site-linker/TFF2 fusion compound is made. In the 5′ to 3′ direction the component DNA sequences are joined by designing suitable restriction sites at the termini of the components using PCR amplification and cutting and ligating the fragments together using commercially available restriction enzymes and DNA ligases, respectively, as is known to one skilled in the art. The components, in order, comprise: yeast GAPDH promoter sequence, a yeast alpha mating factor leader sequence, and a DNA sequence of human epidermal growth factor, encoding the protein in SEQ ID NO: 1. To the 3′ of this is added a DNA sequence encoding the linker containing the enterokinase cleavage site, shown in SEQ ID NO: 2. And finally, the DNA sequence of human trefoil family factor 2 (TFF2, also known as hSP, see Tomassetto, C., EMBO J. 9:407, 1990) is added to the 3′ end, encoding the protein in SEQ ID NO: 3, and finally a yeast alpha mating factor terminator.

The accuracy of the construct is confirmed by DNA sequencing and is then cloned into a commercially available yeast expression vector, Yep24 (from American Type Culture Collection and containing the Ura 3 gene for use as a selectable marker.) This construct is transfected into the production yeast strain, INVScl (from Invitrogen and deficient in Ura 3), using methods known in the art. A selected transformant containing the resultant plasmid, pEET-1, is used for fermentation to produce the fusion compound polypeptide as a largely secreted product.

The transformant is grown is grown in 8 liters of YPD medium plus 60 ng/l yeast extract at 30° C. until an OD 650 of over 50 is reached, as described in Thim, L., et al., FEBS Lett 318:345-52, 1993. The fermentation broth is cleared by centrifugation, concentrated by Amicon filtration, adjusted to pH 1.7, adjusted to a conductivity of 4.5 mS, and loaded and eluted from a Fast Flow S-Sepharose column as described in Thim, L. FEBS Lett 318:345-52, 1993. The fusion protein is detected in the resulting fractions by measuring the activity of neutralized aliquots in a primary rat hepatocyte proliferation assay for EGF as described in Calnan, D., et al. Gut 47:622-27, 2000. Activity in peak fractions is confirmed by SDS-PAGE, whereby fractions containing significant amounts of fusion protein have a band or bands at or near 20 KD MW that is cleaved following incubation of second aliquot in vitro with enterokinase (Stratagene), as described in Gaillard, I., et al., Biochemistry 35:6150, 1996, while contaminating bands are largely unchanged.

Pooled fractions are further purified using endotoxin-free equipment and Vydac C4 reverse phase HPLC column chromatography as described in Thim, L., FEBS Lett 318:345-52,1993. RP-HPLC in acetonitrile and TFA as described will separate glycosylated from unglycosylated fusion protein as well as aggregates and unwanted endotoxin from the desired product. Endotoxin in final pooled material is measured using the limulus amebocyte assay (Associates of Cape Cod, Inc, Woods Hole, Mass.) and is less than 1EU/mg of purified protein. Peak fractions are determined by SDS-PAGE analysis as above, pooled, and diafiltered against 20 mM sodium phosphate buffer (pH 6.8).

Purified material is concentrated by filtration to appropriate concentrations for therapeutic administration, such as about 8 mg protein/ml, in 1% carboxymethyl cellulose (for oral administration) or in 40 mg/ml mannose (for sterile filtration and lyophilization of parenteral drug). Oral doses are stored at −20° C., and lyophilized material is stable at 4° C.

The final preparation passes quality control measurements such as being over 95% pure by reducing SDS-PAGE on a 12% gel, having less than 5% oligomers by non-reducing SDS-PAGE, displaying over 90% of the expected N-terminal sequence by Edman degradation, and having less than 1 EU endotoxin per mg protein, and a biological activity per mole of fusion protein that is within about 20% of the activity of monomeric EGF component when assayed in the EGF bioassay at equimolar concentrations. Similarly, the purified fusion compound has a biological activity within about 20% of the bioactivity of the TFF2 component when assayed at equimolar concentrations in an epithelial cell migration assay in vitro (Poulsom, R., Baillieres Clin Gastroenterol 10:113-34, 1996).

Patients being treated for acute gastrointestinal disorders receive up to about 500 mg per day of fusion compound in oral formulation and up to about 2 mg/kg/day in subcutaneous preparations made up fresh in sterile water for injection.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

### REFERENCES

Ansel, H. C., Allen, L, Popovich, N. G. (eds.) (1999) Pharmaceutical Dosage Forms and Drug Delivery Systems, 7^{th} ed. Lippencott Williams and Wilkins Publishers.

Bashkin, J. K., Sampath, U., Frolova, E. (1995) Ribozyme mimics as catalytic antisense reagents. Appl. Biochem. Biotechnol. 54:43-56.

Beigelman, L., Karpeisky, A., Matulic-Adamic, J., Haeberli, P., Sweedler, D., Usman, N. (1995) Synthesis of 2′-modified nucleotides and their incorporation into hammerhead ribozymes. Nucleic Acids Res. 23:4434-42.

Deutscher, M. P., Simon, M. I., Abelson, J. N. (eds.) (1990) Guide to Protein Purification: Methods in Enzymology (Methods in Enzymology Series, Vol 182), Academic Press.

Gaudilliere, B., Shi, Y., Bonni, A. (2002) RNA interference reveals a requirement for MEF2A in activity-dependent neuronal survival. J. Biol. Chem. 2002 Sep 13; [epub ahead of print].

Gennaro, A. (ed.) (2000) Remington: The Science and Practice of Pharmacy, 20th edition, Lippincott, Williams, & Wilkins.

Gorman, C. M., Merlino, G. T., Willingham, M. C., Pastan, I., Howard, B. H. (1982) The Rous sarcoma virus long terminal repeat is a strong promoter when introduced int a variety of eucaryotic cells by DNA-mediated transfection. Proc. Natl. Acad. Sci. (USA) 79:6777-6781.

Grosschedl R., Baltimore D. (1985) Cell-type specificity of immunoglobulin gene expression is regulated by at least three DNA sequence elements. Cell 41:885-97.

Howard, G. C., Bethell, D. R. (2000) Basic Methods in Antibody Production and Characterization. CRC Press.

Jost, C. R. Kurucz I., Jacobus C. M., Titus J. A., George A. J., Segal D. M. (1994) Mammalian Expression and Secretion of Functional Single-Chain Fv Molecules. J. Biol. Chem. 269:26267-73.

Kabat et al. (1991) Sequences of Proteins of Immunological Interest, N.I.H. publication no. 91-3242.

Kibbe, A. H. (ed.) (2000) Handbook of Pharmaceutical Excipients 3^{rd} ed. Amer. Pharmaceutical Assoc.

Liu A. Y., Robinson R. R., Murray E. D. Jr., Ledbetter J. A., Hellstrom I., Hellstrom K. E. (1987) Production of a mouse-human chimeric monoclonal antibody to CD20 with potent Fc-dependent biologic activity J. Immunol. 139:3521-6.

Liu A. Y., Robinson R. R., Hellstrom K. E., Murray E. D. Jr., Chang C. P., Hellstrom I. (1987) Chimeric Mouse-Human IgG1 Antibody that can Mediate Lysis of Cancer Cells, Proc. Natl. Acad. Sci. USA 84: 3439-43.

Milligan, J. F., Matteucci, M. D., Martin, J. C. (1993) Current concepts in antisense drug design. J. Med. Chem. 36:1923-1937.

Okayama H, Berg P. (1983) A cDNA cloning vector that permits expression of cDNA inserts in mammalian cells. Mol. Cell. Bio. 3:280-9.

O'Neil, N. J., Martin, R. L., Tomlinson, M. L., Jones, M. R., Coulson, A., Kuwabara, P. E. (2001) RNA-mediated interference as a tool for identifying drug targets. Am. J. Pharmacogenomics 1:45-53.

Remington, J. P. (1985) Remington's Pharmaceutical Sciences, 17th edition, Mack Publishing Company.

Wagner R W, Matteucci M D, Lewis J G, Gutierrez A J, Moulds C, Froehler B C. (1993) Antisense gene inhibition by oligonucleotides containing C-5 propyne pyrimidines. Science 260:1510-1513.

Wagner, R. W., Matteucci, M. D., Grant, D., Huang, T., Froehler, B. C. (1996) Potent and selective inhibition of gene expression by an antisense heptanucleotide. Nat. Biotechnol. 14:840-844.

TABLE 2

Brief Description of the Sequences.

| | |
|---|---|
| Description | SEQ ID NO |

| | |
|---|---|
| forward PCR primer for REB BAC DNA: | 3 |
| 5′-CTGATATGTGCCCATGTTCCAAAC-3′ | |
| reverse PCR primer for REB BAC DNA: | 4 |
| 5′-CCTTGCTGAATGCAGATGTTTCAC-3′ | |
| NOS/rv PCR primer | 5 |
| CGGCAACAGGATTCAATCT | |

What is claimed is:

1. A method of delivery of a plurality of component molecules to a multi-cellular host, comprising the steps of:
(a) providing a composition comprising a chimeric molecule; and
(b) administering the chimeric molecule to the host to produce a treated host,
wherein the chimeric molecule comprises at least one first component molecule, at least one linker, and at least one second component molecule; wherein the linker comprises an enzyme cleavage site and wherein at least a first linker is operably linked to a first component molecule and a second component molecule to produce a non-naturally occurring linkage and cleavage site between the first component molecule and second component molecule;
wherein the cleavage site is engineered for cleavage in vivo by a host enzyme and is not susceptible to cleavage in a production host;
wherein, upon cleavage of the chimeric molecule at the cleavage site, at least one of the component molecules is functionally active; and
wherein at least one of the first and second component molecules comprises one selected from the group consisting of a peptide, a protein, or an active fragment thereof.

2. The method of claim 1, wherein the cleavage site is engineered for cleavage in vivo by an enzyme in the gastrointestinal tract of the host.

3. The method of claim 1, wherein the enzyme is enterokinase, a matrix metalloproteinase, or a tissue type plasminogen activator.

4. The method of claim 1, wherein upon cleavage of the chimeric molecule at the enzyme cleavage site, at least two of the component molecules are functionally active.

5. The method of claim 1, wherein at least one of the component molecules is functionally active prior to cleavage of the chimeric molecule.

6. The method of claim 1, wherein the component molecules are non-inhibitory molecules.

7. The method of claim 1, wherein the component molecules are non-cytotoxic molecules.

8. The method of claim 1, wherein the first component molecule is the same as the second component molecule.

9. The method of claim 1, wherein the chimeric molecule has a formula: A(xᵢBᵢ)ⁿ, wherein A represents the first component molecule, x represents the linker, B represents the second component molecule, i and n are each a positive integer.

10. The method of claim 9, wherein the formula is selected from the group consisting of:
(a) A(x₁B₁);
(b) A(x₁B₁)(x₂B₂), wherein x₁ and x₂ may be the same or different, and B₁ and B₂ may be the same or different;
(c) A(x₁B₁)(x₂B₂)(x₃B₃), wherein x₁, x₂ and x₃ may each be the same or different, and B₁, B₂ and B₃ may each be the same or different;
(d) A(x₁B₁)(x₂B₂)(x₃B₃)(x₄B₄), wherein x₁, x₂, x₃ and x₄ may each be the same or different, and B₁, B₂, B₃ and B₄ may each be the same or different; and
(e) A(x₁B₁)(x₂B₂)(x₃B₃)(x₄B₄)(x₅B₅), wherein x₁, x₂, x₃, x₄ and x₅ may each be the same or different, and B₁, B₂, B₃, B₄ and B₅ may each be the same or different.

11. The method of claim 1, wherein the first component molecule is a peptide or protein or an active fragment thereof and at least one second component molecule is selected from the group consisting of: peptides, proteins, nucleic acids, carbohydrates, synthetic polymers, plant products, fungal products, small molecule drugs, detectable molecules, haptens, ligands, anti-infectives, and analogs and fragments thereof.

12. The method of claim 1, wherein the chimeric molecule is a polyprotein.

13. The method of claim 9, wherein the chimeric molecule is a polyprotein.

14. The method of claim 10, wherein the chimeric molecule is a polyprotein.

15. The method of claim 10, wherein x₁, x₂, x₃, x₄ and x₅ are the same.

16. The method of claim 10, wherein B₁, B₂, B₃, B₄ and B₅ are the same.

17. The method of claim 1, wherein at least one of the component molecules is selected from the group consisting of: antigens, soluble receptors, growth factors, cytokines, lymphokines, chemokines, enzymes, anti-infectives, prodrugs, toxins, and active fragments thereof.

18. The method of claim 1, wherein at least one of the component molecules is selected from the group consisting of: soluble p75TNFα receptor Fc fusion, human growth hormone, granulocyte colony stimulating factor (GCSF), granulocyte-macrophage colony stimulating factor (GM-CSF), interferon-α2b, pegylated (PEG) interferon-α, PEG-asparagase, PEG-adamase, anti-CO 17-1 A, hirudin, tissue type plasminogen activator, erythropoietin, human DNAase, IL-2, coagulation factor IX, IL-11, TNKase, activated protein C, PDGF, coagulation factor VIa, insulin, interferon α-N3, interferon γ 1b, interferon α consensus sequence, platelet activating factor acetyl hydrolase and active fragments thereof.

19. The method of claim 1, wherein the first component molecule is a peptide, protein or an active fragment thereof and the second component molecule is a chemical compound.

20. The method of claim 1, wherein at least one of the component molecules is an antibody.

21. The method of claim 1, wherein first component molecule is an antibody or an active fragment thereof and the second component molecule is other than an antibody.

22. The method of claim 1, wherein second component molecule is an antibody or an active fragment thereof and the first component molecule is other than an antibody.

23. The method of claim 1, wherein the first and second component molecules are each an antibody or an active fragment thereof.

24. The method of claim 1, wherein at least one of the component molecules is selected from the group consisting of anti-microbial peptides, proteins, analogs or active fragments thereof.

25. The method of claim 1, wherein at least one of the component molecules is a defensin, a lysozyme, or a lactoferrin.

26. The method of claim 1, wherein at least one of the component molecules is selected from the group consisting of human and non-human animal peptides, proteins, analogs and active fragments thereof.

27. The method of claim 1, wherein at least one of the component molecules is selected from the group consisting of plant peptides, proteins, analogs or active fragments thereof.

28. The method of claim 9, wherein at least one of the component molecules is selected from the group consisting of microbial peptides, proteins, analogs or active fragments thereof.

29. The method of claim 1, wherein at least one of the component molecules is selected from the group consisting of fish peptides, proteins, analogs or active fragments thereof.

30. The method of claim 9, wherein at least two of the components are selected from the group consisting of: peptides, proteins, analogs or active fragments thereof.

31. The method of claim 1, wherein the peptide or protein is selected from the group consisting of: IGF-I, EGF, PDGF, ITF, KGF, lactoferrin, lysozyme, fibrinogen, α₁-antitrypsin, erythropoietin, hGH, tPA, interferon alpha, interferon beta, interferon gamma, consensus interferon, insulin, human chorionic gonadotropin, diphtheria protein, and anti-hemophilic factor.

32. The method of claim 1, wherein at least one of the component molecules is a hormone.

33. The method of claim 32, wherein the hormone is selected from the group consisting of: testosterone, estrogen, and progesterone.

34. The method of claim 1, wherein at least one of the component molecules is selected from the group consisting of: taxol or its analogs or derivatives, matrix metalloproteinase inhibitors, and anti-infectives.

35. The method of claim 9, wherein at least two of the component molecules are selected from the group consisting of the combinations: lactoferrin/lactoferrin; lactoferrin/lysozyme; lysozyme/lysozyme; lactoferrin/EGF; EGF/EFG; lactoferrin/ITF; ITF/ITF; ITF/EFG; EGF/KGF; KGF/KGF; ITF/KGF; KGF/PDGF; PDGF/PDGF; α₁-antitrypsin/MMP inhibitor; estrogen/progesterone; antibody/antibody; ITF/ITF; and analogs, variants and derivatives thereof.

36. The method of claim 1, wherein administration of the chimeric molecule achieves a biological effect in the treated host, and the biological effect is diagnostic, prophylactic, therapeutic, anti-infective or nutritional.

37. The method of claim 1, wherein the chimeric molecule further comprises at least a fragment of an additional polypeptide, wherein the polypeptide is highly expressed in the production host.

38. The method of claim 1, wherein the cleavage site is engineered for cleavage in vivo, extracellularly in the treated host, other than at a cell surface.

39. The method of claim 38, wherein neither the first nor the second component molecule is interferon-beta.

40. The method of claim 1, wherein the cleavage site is engineered for cleavage in vivo in the treated host at a cell surface.

41. The method of claim 1, wherein the first component molecule is not an antibody or an antibody fragment.

42. The method of claim 1, wherein the cleavage site is engineered for cleavage by an endogenous treated host enzyme.

43. The method of claim 1, wherein the cleavage site is engineered for cleavage by an endogenous host enzyme selected from the group consisting of: coagulation factors; ADAMTS 4, 5; Aggreganases 1, 2; thrombin; plasmin; complement factors; gastricin; granule proteases; matrix metalloproteinases; membrane type matrix metalloproteinases; type II transmembrane serine proteases; ADAMs; neprilysin; tissue-type plasminogen activator, and caspases.

44. The method of claim 1, wherein the cleavage site is engineered for cleavage in vivo intracellularly by an enzyme in the treated host, and the combination of first and second component molecules is other than the combination of a protein transduction domain and a cytotoxic domain.

45. The method of claim 1, wherein the cleavage site is engineered for cleavage in vivo intracellularly by an enzyme in the treated host, and the cleavage site is not a viral pathogen activated cleavage site.

46. The method of claim 1, wherein the cleavage site is engineered for cleavage in vivo intracellularly by an enzyme in the treated host, and the second component is not a cytotoxic molecule.

47. The method of claim 1, wherein the chimeric molecule further comprises a leader sequence for directing secretion of the chimeric molecule in a production host or for directing storage of the chimeric molecule in the production host.

48. The method of claim 1, wherein the chimeric molecule comprises a targeting molecule for directing the chimeric molecule to a location for action in the treated host.

49. The method of claim 1, wherein the chimeric molecule further comprises a purification moiety that facilitates in vitro purification of the chimeric molecule after production from a production host.

50. The method of claim 1, wherein the linker comprises two cleavage sites and a spacer adjacent between the two cleavage sites.

51. The method of claim 1, wherein the chimeric molecule is a component of an edible product.

52. The method of claim 51, wherein the edible product is selected from the group consisting of milk, a plant, a seed, a microbial cell, and derivatives and extracts thereof.

53. The method of claim 51, wherein the edible product is a cereal grain.

54. The method of claim 1, wherein the chimeric molecule is administered orally, parenterally, or by inhalation.

55. The method of claim 1, wherein the chimeric molecule is administered parenterally by intravenous route, subcutaneous route, intraperitoneal route, intracardiac route, or transdermal route.

56. The method of claim 1, wherein the chimeric molecule is not a nucleic acid molecule.

57. The method of claim 1, wherein the chimeric molecule further comprises an additional molecule that is linked to the first component molecule, but not to the linker, wherein the additional molecule is highly expressed in the production host.

58. The method of claim 1, wherein at least one of the first or second component molecules is an antibody or an active fragment thereof and the antibody is selected from the group consisting of: anti-IL8, anti-CD11a, anti-ICAM-3, anti-CD80, anti-CD2, anti-CD3, anti-complement C5, anti-TNFα, anti-CD4, anti-α4β7, anti-CD40L (ligand), anti-VLA4, anti-CD64, anti-IL5, anti-IL4, anti-IgE, anti-CD23, anti-CD147, anti-CD25, anti-β2 integrin, anti-CD 18, anti-TGF#2, anti-Factor VII, anti-II_{b}IIₐ receptor, anti-PDGFβR, anti-F protein (from RSV), anti-gp120 (from HIV), anti-Hep B, anti-CMV, anti-CD14, anti-VEFG, anti-CA125 (ovarian cancer), anti-17-1A (colorectal cell surface antigen), anti-anti-idiotypic GD3 epitope, anti-EGFR, anti-HER2/neu; anti-αVβ3 integrin, anti-CD52, anti-CD33, anti-CD20, anti-CD22, anti-HLA, and anti-HLA DR or an active fragment thereof.

59. The method of claim 1, wherein the composition further comprises a pharmaceutically acceptable carrier or excipient.

60. A kit comprising a composition comprising a chimeric molecule and a package insert comprising instructions for administration of composition to a human or non-human animal treated host, wherein the chimeric molecule comprises at least one first component molecule, at least one linker, and at least one second component molecule; wherein the linker comprises an enzyme cleavage site and wherein at least a first linker is operably linked to a first component molecule and a second component molecule to produce a non-naturally occurring linkage and cleavage site between the first component molecule and second component molecule;
wherein the cleavage site is engineered for cleavage in vivo by a treated host enzyme and is resistant to cleavage in any production host;
wherein, upon cleavage of the chimeric molecule at the cleavage site, at least one of the component molecules is functionally active; and
wherein at least one of the first and second component molecules comprises one selected from the group consisting of a peptide, a protein, or an analog or active fragment or derivative thereof.

61. The kit of claim 60, wherein the cleavage site is engineered for cleavage in vivo in the gastrointestinal tract of the treated host.

62. The kit of claim 60, wherein the cleavage site is engineered for cleavage in vivo by enterokinase.

63. The kit of claim 60, wherein the cleavage site is engineered for cleavage in vivo extracellularly in the treated host, other than at a cell surface.

64. The kit of claim 60, wherein the cleavage site is engineered for cleavage in vivo in the treated host, at a cell surface.

65. The kit of claim 60, wherein the cleavage site is engineered for cleavage in vivo intracellularly in the treated host by an endogenous host enzyme.

66. The kit of claim 65, wherein the combination of the first component molecule and second component molecule is not a combination of a protein transduction domain and a cytotoxic domain.

67. The kit of claim 60, wherein the cleavage site is engineered for cleavage in vivo intracellularly in the treated host, and wherein the cleavage site is not a viral pathogen activated cleavage site.

68. The kit of claim 60, wherein the cleavage site is engineered for cleavage in vivo intracellularly in the treated host, and the second component molecule is other than a cytotoxic molecule.

69. A chimeric molecule that comprises a formula: A(xᵢBᵢ)ⁿ, wherein A represents the first component molecule, x represents the linker, B represents the second component molecule, i and n are each a positive integer, and wherein the chimeric molecule comprises at least one first component molecule, at least one linker, and at least one second component molecule; wherein the linker comprises an enzyme cleavage site and wherein at least a first linker is operably linked to a first component molecule and a second component molecule to produce a non-naturally occurring linkage and cleavage site between the first component molecule and second component molecule;
wherein the cleavage site is engineered for cleavage in vivo by a host enzyme and is not susceptible to cleavage in a production host;
wherein, upon cleavage of the chimeric molecule at the cleavage site, at least one of the component molecules is functionally active; and
wherein at least one of the first and second component molecules comprises one selected from the group consisting of a peptide, a protein, or an analog or active fragment or derivative thereof.

70. The chimeric molecule of claim 69, wherein the formula is selected from the group consisting of:
(a) A(x₁B₁);
(b) A(x₁B₁)(x₂B₂), wherein x, and x₂ may be the same or different, and B₁ and B₂ may be the same or different;
(c) A(x₁B₁)(x₂B₂)(x₃B₃), wherein x₁, x₂ and x₃ may each be the same or different, and B₁, B₂ and B₃ may each be the same or different;
(d) A(x₁B₁)(x₂B₂)(x₃B₃)(x₄B₄), wherein x₁, x₂, x₃ and x₄ may each be the same or different, and B₁, B₂, B₃ and B₄ may each be the same or different; and
(e) A(x₁B₁)(x₂B₂)(x₃B₃)(x₄B₄)(x₅Bs), wherein x₁, x₂, x₃, x₄ and x₅ may each be the same or different, and B₁, B₂, B₃, B₄ and B₅ may each be the same or different.

71. The chimeric molecule of claim 69, wherein the chimeric molecule is a polyprotein.

72. A nucleic acid molecule encoding the chimeric molecule of claim 71.

73. A vector comprising the nucleic acid molecule of claim 72.

74. A host cell comprising the nucleic acid molecule of claim 72.

75. A method for the preparation of a chimeric molecule in a production host for administration to a treated host comprising: (a) providing a nucleic acid molecule that encodes a chimeric molecule; (b) transforming a production host with the nucleic acid molecule; (c) allowing the production host to produce the chimeric molecule; (d) recovering the chimeric molecule from the production host; and (e) performing quality control on the harvested chimeric molecule to meet regulatory approval;
wherein the chimeric molecule comprises component molecules including a first component molecule, a linker that comprises a cleavage site, and a second component molecule,
wherein at least one of the first and second component molecules comprises a peptide, a protein or an active fragment thereof;
wherein the linker is operably linked to the first and second component molecules to produce a non-naturally occurring linkage and cleavage site;
wherein the cleavage site is engineered for in vivo cleavage by a treated host enzyme.

76. The method of claim 75, wherein the enzyme is present in the gastrointestinal tract of the treated host.

77. The method of claim 75, wherein the enzyme is an enzyme that acts extracellularly in the treated host, but not at a cell surface.

78. The method of claim 75, wherein the enzyme is an enzyme that acts at a cell surface in the treated host.

79. The method of claim 75, wherein the enzyme is an enzyme that acts intracellularly in the treated host.

80. The method of claim 79, wherein the chimeric molecule is other than a combination of a protein transduction domain and a cytotoxic domain.

81. The method of claim 75, wherein the enzyme is an enzyme that acts intracellularly in the treated host and the cleavage site is not a viral pathogen activated cleavage site.

82. The method of claim 75, wherein the enzyme is an enzyme that acts intracellularly in the treated host and the second component molecule is other than a cytotoxic molecule.

83. The method of 75, wherein the production host is selected from the group consisting of: a bacterial cell, a fungal cell, a mammalian cell, a plant cell, a plant seed, an insect cell, a plant, a fungus, and an animal.

84. A composition comprising a chimeric molecule and a pharmaceutically acceptable carrier for administration to a treated host, wherein the chimeric molecule
comprises component molecules including at least one first component molecule, at least one linker, and at least one second component molecule; wherein the linker comprises an enzyme cleavage site and wherein at least a first linker is operably linked to a first component molecule and a second component molecule to produce a non-naturally occurring linkage and cleavage site between the first component molecule and second component molecule;
wherein the cleavage site is engineered for cleavage in vivo by a treated host enzyme and is resistant to cleavage in a production host;
wherein, upon cleavage of the chimeric molecule at the cleavage site, at least one of the component molecules is functionally active; and
wherein at least one of the first and second component molecules comprises one selected from the group consisting of a peptide, a protein, or an active fragment thereof.

85. The composition of claim 84, wherein the cleavage site is engineered for in vivo cleavage by an enzyme in the gastrointestinal tract of the treated host.

86. The composition of claim 84, wherein the enzyme is enterokinase.

87. The composition of claim 84, wherein the cleavage site is engineered for in vivo cleavage by an enzyme in an inflammatory tissue of the treated host.

88. The composition of claim 87, wherein the inflammatory tissue is inflammatory bowel or synovium.

89. The composition of claim 84, wherein the cleavage site is engineered for in vivo cleavage extracellularly in the treated host other than at a cell surface.

90. The composition of claim 84, wherein the cleavage site is engineered for in vivo cleavage in the treated host at a cell surface.

91. The composition of claim 84, wherein the cleavage site is engineered for in vivo cleavage intracellularly in the treated host by an endogenous treated host enzyme.

92. The composition of claim 84, wherein the cleavage site is engineered for in vivo cleavage intracellularly in the treated host and wherein the combination of the first and second component molecules is not a combination of a protein transduction domain and a cytotoxic domain.

93. The composition of claim 84, wherein the cleavage site is engineered for in vivo cleavage intracellularly in the treated host and wherein the second component molecule is not a cytotoxic molecule.

94. The composition of claim 84, wherein the composition is encapsulated.

95. The composition of claim 84, wherein one of the component molecules binds to an extracellular matrix in the treated host.

96. The composition of claim 84, wherein the chimeric molecules comprises two cleavage sites, one of which is engineered for cleavage in vitro after expression is a production host and the other is engineered for cleavage in vivo in the treated host.

97. The composition of claim 84, wherein the composition is formulated for oral delivery.

98. The composition of claim 84, wherein the composition is formulated for parenteral delivery.

99. The composition of claim 98, wherein parenteral delivery is selected from the group consisting of: subcutaneous, intravenous, intra-arterial, intraventricular, intracranial, percutaneous and transdermal delivery

100. The composition of claim 84, wherein the composition is formulated for intranasal delivery or for inhalation.

101. The composition of claim 84, wherein the chimeric molecule is a vaccine.

102. The composition of claim 84, wherein the chimeric molecule comprises an adjuvant as one of the component molecules.

103. The composition of claim 101, wherein the vaccine comprises a component of a pathogenic organism.

104. The composition of claim 101, wherein the vaccine is a cancer vaccine, and the component molecules are molecules that are over-expressed in a cancer cell.

105. The use of a chimeric molecule in the preparation of a medicament for diagnosis, prophylaxis, treatment of a disease or condition, or for enhancement of nutrition in a subject in need of such, wherein the chimeric molecule comprises at least one first component molecule, at least one linker, and at least one second component molecule; wherein the linker comprises an enzyme cleavage site and wherein at least a first linker is operably linked to a first component molecule and a second component molecule to produce a non-naturally occurring linkage and cleavage site between the first component molecule and second component molecule; wherein the cleavage site is engineered for cleavage in vivo by a treated host enzyme and is not susceptible to cleavage in a production host; wherein, upon cleavage of the chimeric molecule at the cleavage site, at least one of the component molecules is functionally active; and wherein at least one of the first and second component molecule comprises one selected from the group consisting of a peptide, a protein, or an active fragment thereof.


SEQUENCE LISTING (8 sequences)

SEQ ID NO: 1 - protein, 53 aa, Artificial
  human epidermal growth factor
NSDSECPLSHDGYCLHDGVCMYIEALDKYACNCVVGYIGERCQYRDLKWWELR

SEQ ID NO: 2 - protein, 8 aa, Artificial
  linker containing the enterokinase cleavage site
GGGDDDDK

SEQ ID NO: 3 - protein, 106 aa, Artificial [sequence omitted]

SEQ ID NO: 4 - DNA, 24 bp, Artificial
  reverse PCR primer for REB BAC DNA
ccttgctgaatgcagatgtttcac

SEQ ID NO: 5 - DNA, 19 bp, Artificial
  NOS/rv PCR primer
cggcaacaggattcaatct

SEQ ID NO: 6 - DNA, 24 bp, Artificial
  forward PCR primer for REB BAC DNA
ctgatatgtgcccatgttccaaac

SEQ ID NO: 7 - protein, 16 aa, Artificial
  spacer 1
ASGGGGIEGRGGGGSA

SEQ ID NO: 8 - protein, 6 aa, Artificial
  spacer 2
GGRSGG